SEQUENCE LISTING (13 sequences)

SEQ ID NO: 1 - protein, 360 aa, Ovis aries
MKATIIFFLVAQVSWAGPFQQKGLFDFMLEDEASGIGPEERFHEVPELEPMGPVCPFRCQCHLRVVQCSDLGLEKVPKDLPPDTALLDLQNNKITEIKDGDFKNLKNLHTLILINNKISKISPGAFAPLVKLERLYLSKNQLKELPEKMPKTLQELRVHENEITKVRKSVFNGLNQMIVVELGTNPLKSSGIENGAFQGMKKLSYIRIADTNITTIPQGLPPSLTELHLDGNKITKVDAASLKGLNNLAKLGLSFNSISAVDNGSLANTPHLRELHLNNNKLVKVPGGLADHKYIQVVYLHNNNISAIGSNDFCPPGYNTKKASYSGVSLFSNPVQYWEIQPSTFRCVYVRAAVQLGNYK

SEQ ID NO: 2 - protein, 140 aa, Ovis aries
DEASGIGPEERFHEVPELEPMGPVCPFRCQCHLRVVQCSDLGLEKVPKDLPPDTALLDLQNNKITEIKDGDFKNLKNLHTLILINNKISKISPGAFAPLVKLERLYLSKNQLKELPEKMPKTLQELRVHENEITKVRKSV

SEQ ID NO: 3 - protein, 147 aa, Ovis aries
DEASGIGPEERFHEVPELEPMGPVCPFRCQCHLRVVQCSDLGLEKVPKDLPPDTALLDLQNNKITEIKDGDFKNLKNLHTLILINNKISKISPGAFAPLVKLERLYLSKNQLKELPEKMPKTLQELRVHENEITKVRKSVFNGLNQM

SEQ ID NO: 4 - protein, 158 aa, Ovis aries
DEASGIGPEERFHEVPELEPMGPVCPFRCQCHLRVVQCSDLGLEKVPKDLPPDTALLDLQNNKITEIKDGDFKNLKNLHTLILINNKISKISPGAFAPLVKLERLYLSKNQLKELPEKMPKTLQELRVHENEITKVRKSVFNGLNQMIVVELGTNPLK

SEQ ID NO: 5 - protein, 359 aa, Homo sapiens
MKATIILLLLAQVSWAGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 6 - protein, 139 aa, Homo sapiens
DEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVT

SEQ ID NO: 7 - protein, 157 aa, Homo sapiens
DEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLK

SEQ ID NO: 8 - protein, 354 aa, Mus musculus
MKATLIFFLLAQVSWAGPFEQRGLFDFMLEDEASGIIPYDPDNPLISMCPYRCQCHLRVVQCSDLGLDKVPWDFPPDTTLLDLQNNKITEIKEGAFKNLKDLHTLILVNNKISKISPEAFKPLVKLERLYLSKNQLKELPEKMPRTLQELRVHENEITKLRKSDFNGLNNVLVIELGGNPLKNSGIENGAFQGLKSLSYIRISDTNITAIPQGLPTSLTEVHLDGNKITKVDAPSLKGLINLSKLGLSFNSITVMENGSLANVPHLRELHLDNNKLLRVPAGLAQHKYIQVVYLHNNNISAVGQNDFCRAGHPSRKASYSAVSLYGNPVRYWEIFPNTFRCVYVRSAIQLGNYK

SEQ ID NO: 9 - protein, 134 aa, Mus musculus
DEASGIIPYDPDNPLISMCPYRCQCHLRVVQCSDLGLDKVPWDFPPDTTLLDLQNNKITEIKEGAFKNLKDLHTLILVNNKISKISPEAFKPLVKLERLYLSKNQLKELPEKMPRTLQELRVHENEITKLRKSD

SEQ ID NO: 10 - protein, 152 aa, Mus musculus
DEASGIIPYDPDNPLISMCPYRCQCHLRVVQCSDLGLDKVPWDFPPDTTLLDLQNNKITEIKEGAFKNLKDLHTLILVNNKISKISPEAFKPLVKLERLYLSKNQLKELPEKMPRTLQELRVHENEITKLRKSDFNGLNNVLVIELGGNPLK

SEQ ID NO: 11 - protein, 360 aa, Bos taurus
MKATIIFLLVAQVSWAGPFQQKGLFDFMLEDEASGIGPEEHFPEVPEIEPMGPVCPFRCQCHLRVVQCSDLGLEKVPKDLPPDTALLDLQNNKITEIKDGDFKNLKNLHTLILINNKISKISPGAFAPLVKLERLYLSKNQLKELPEKMPKTLQELRVHENEITKVRKSVFNGLNQMIVVELGTNPLKSSGIENGAFQGMKKLSYIRIADTNITTIPQGLPPSLTELHLDGNKITKVDAASLKGLNNLAKLGLSFNSISAVDNGSLANTPHLRELHLNNNKLVKVPGGLADHKYIQVVYLHNNNISAIGSNDFCPPGYNTKKASYSGVSLFSNPVQYWEIQPSTFRCVYVRAAVQLGNYK

SEQ ID NO: 12 - protein, 140 aa, Bos taurus
DEASGIGPEEHFPEVPEIEPMGPVCPFRCQCHLRVVQCSDLGLEKVPKDLPPDTALLDLQNNKITEIKDGDFKNLKNLHTLILINNKISKISPGAFAPLVKLERLYLSKNQLKELPEKMPKTLQELRVHENEITKVRKSV

SEQ ID NO: 13 - protein, 158 aa, Bos taurus
DEASGIGPEEHFPEVPEIEPMGPVCPFRCQCHLRVVQCSDLGLEKVPKDLPPDTALLDLQNNKITEIKDGDFKNLKNLHTLILINNKISKISPGAFAPLVKLERLYLSKNQLKELPEKMPKTLQELRVHENEITKVRKSVFNGLNQMIVVELGTNPLK

(19) United States
(12) Patent Application Publication
(10) Pub. No.: US 2026/0216282 A1
(43) Pub. Date: Jul. 30, 2026

(54) METHODS AND AGENTS FOR TREATING TISSUE ISCHEMIA AND ASSOCIATED DISEASES AND CONDITIONS

(71) Applicant: AROA BIOSURGERY LIMITED, Auckland (NZ)

(72) Inventors: Barnaby MAY, Auckland (NZ); Robert VEALE, Auckland (NZ); Sandi DEMPSEY, Auckland (NZ)

(21) Appl. No.: 19/143,759

(22) PCT Filed: Jan. 24, 2024
(86) PCT No.: PCT/NZ2024/050005

§ 371 Date: Jun. 26, 2025

(30) Foreign Application Priority Data
Jan. 27, 2023 (AU) 2023900186

(51) Int. Cl. A61K 38/17; A61P 9/10

(52) U.S. Cl. CPC A61K 38/1709; A61P 9/10

(57) ABSTRACT

The present invention is directed to the use of MayDay peptide fragments, named Decorin31-170, Decorin31-188, Decorin31-177, Decorin31-169 or Decorinl-188 (Decorin protein N-terminal fragments) in the treatment and prevention of tissue ischemia and diseases associated with ischemia such as myocardial infarction (MI), reperfusion injury, organ transplantation, wound healing, stroke, cardiomyopathy and heart failure. Also anticipated in the present invention is methods of modulating tissue repair and modulating stem cell recruitment.

### TECHNICAL FIELD

The invention relates to methods of treating or preventing a disease or condition by administration of a bioactive MayDay polypeptide. More particularly, the invention relates to methods of treating or preventing tissue ischemia and/or a disease or condition associated with tissue ischemia by administration of a MayDay polypeptide as described herein.

### BACKGROUND OF THE INVENTION

The following includes information that may be useful in understanding the present inventions. It is not an admission that any of the information provided herein is prior art, or relevant, to the presently described or claimed inventions, or that any publication or document that is specifically or implicitly referenced is prior art. Any discussion of the prior art throughout the specification should in no way be considered as an admission that such prior art is widely known or forms part of the common general knowledge in the field.

While significant progress has been made in the treatment and in some cases prevention of a substantial number of diseases, many diseases and conditions remain challenging to treat or indeed have no effective treatments available. In other cases, treatments may be available, but attendant with their use are one or more undesirable side effects. For example, certain diseases and conditions in which the subject's immune system is involved remain challenging to effectively treat, as treatment modalities can have adverse effects on otherwise beneficial or important aspects of the immune response, such as the use of immunosuppressants and the associated elevated susceptibility to infectious diseases.

As such, the treatment or prevention of various diseases and conditions, such as the treatment or prevention of tissue ischemia and/or one or more diseases or conditions associated with tissue ischemia, remains problematic.

There is a need to develop new and improved methods of treating tissue ischemia and/or diseases or conditions associated with tissue ischemia, such as the development of therapeutic agents suitable for use in such treatments.

The present invention seeks to provide one or more methods of treating or preventing tissue ischemia and/or a disease or condition associated with tissue ischemia, for example by administration of a MayDay polypeptide as disclosed herein, and/or to least provide a useful alternative to existing methods, and/or to at least provide the public with a useful choice.

### SUMMARY OF THE INVENTION

In a first aspect, the invention relates to a method of treating or preventing tissue ischemia and/or a disease or condition associated with tissue ischemia in a subject in need thereof, the method comprising administering to the subject a therapeutically effective amount of a MayDay polypeptide as described herein.

In one example, the invention relates to a method of treating or preventing a disease or condition associated with tissue ischemia in a subject in need thereof, the method comprising administering to the subject a therapeutically effective amount of a MayDay polypeptide as described herein.

In various examples, the disease or condition associated with tissue ischemia is a disease or condition selected from the group consisting of: ischemic-reperfusion injury in an organ for transplantation, spinal cord injury, myocardial infarction, heart failure, acute kidney injury, renal failure, ischemic stroke, hemorrhagic stroke, acute tubular necrosis, compartment syndrome, coronary artery disease, intestinal ischemia, peripheral artery disease, transient ischemic attack, ischemia due to atherosclerosis, chronic kidney disease, pancreatic ischemia, critical limb ischemia, chronic wounds, cirrhosis, liver failure, liver ischemia, vascular dementia, cutaneous ischemia, ischemia due to thrombosis, ischemia due to embolism, ischemia due to trauma, ischemia due to aneurysm, ischemic cardiomyopathy, and traumatic brain injury.

In one particular example, the disease or condition is selected from the group consisting of: myocardial infarction, heart failure, ischemic cardiomyopathy, and stroke.

In one particular example, the disease or condition is selected from the group consisting of: acute kidney injury, acute kidney disease, renal failure, and chronic wounds.

In another aspect, the invention relates to a method of treating or preventing myocardial infarction, heart failure, and/or cardiomyopathy in a subject in need thereof, the method comprising administering to the subject a therapeutically effective amount of a MayDay polypeptide as described herein.

In various examples, the MayDay polypeptide is an isolated, purified, recombinant or synthetic polypeptide selected from the group comprising:
a) a polypeptide comprising, consisting essentially of, or consisting of an amino acid sequence corresponding to residues 1 to 188 of mammalian Decorin;
b) a polypeptide comprising, consisting essentially of, or consisting of an amino acid sequence corresponding to residues 31 to 188 of mammalian Decorin;
c) an N-terminal fragment of mammalian Decorin comprising, consisting essentially of, or consisting of at least 10 contiguous amino acids corresponding to any amino acid sequence within residues 1 to 188 of mammalian Decorin;
d) an N-terminal fragment of mammalian Decorin comprising, consisting essentially of, or consisting of at least 10 contiguous amino acids corresponding to any amino acid sequence within residues 31 to 188 of mammalian Decorin;
e) a polypeptide comprising, consisting essentially of, or consisting of an amino acid sequence corresponding to residues 1 to 177 of mammalian Decorin;
f) a polypeptide comprising, consisting essentially of, or consisting of an amino acid sequence corresponding to residues 31 to 177 of mammalian Decorin;
g) an N-terminal fragment of mammalian Decorin comprising, consisting essentially of, or consisting of at least 10 contiguous amino acids corresponding to any amino acid sequence within residues 1 to 177 of mammalian Decorin;
h) an N-terminal fragment of mammalian Decorin comprising, consisting essentially of, or consisting of at least 10 contiguous amino acids corresponding to any amino acid sequence within residues 31 to 177 of mammalian Decorin;
i) a polypeptide comprising, consisting essentially of, or consisting of an amino acid sequence corresponding to residues 1 to 170 of mammalian Decorin;
j) a polypeptide comprising, consisting essentially of, or consisting of an amino acid sequence corresponding to residues 31 to 170 of mammalian Decorin;
k) a polypeptide comprising, consisting essentially of, or consisting of an amino acid sequence corresponding to residues 31 to 169 of human Decorin;
l) an N-terminal fragment of mammalian Decorin comprising, consisting essentially of, or consisting of at least 10 contiguous amino acids corresponding to any amino acid sequence within residues 1 to 170 of mammalian Decorin;
m) an N-terminal fragment of mammalian Decorin comprising, consisting essentially of, or consisting of at least 10 contiguous amino acids corresponding to any amino acid sequence within residues 31 to 170 of mammalian Decorin;
n) a polypeptide comprising, consisting essentially of, or consisting of the amino acid sequence depicted in any one of Sequence ID No.s: 1 to 13;
o) a polypeptide comprising, consisting essentially of, or consisting of at least about 10 contiguous amino acids from any one of a) to n) above;
p) a polypeptide comprising or consisting of at least about 10 contiguous amino acids from any one of a) to o) above, wherein said polypeptide comprises a motif or region capable of interacting with or recruiting one or more cells, including one or more stem cells;
q) a polypeptide comprising or consisting of at least about 10 contiguous amino acids from any one of a) to p) above, wherein said polypeptide comprises a motif or region capable of interacting with or recruiting one or more mesenchymal stem cells;
r) a functional fragment, a functional variant, a peptide analogue or peptidomimetic, or a derivative of any one of a) to q) above; and
s) a polypeptide having at least about 90% amino acid identity to any one of a) to r) above.

Any of the examples described herein can relate to any of the aspects presented herein.

In one example, the polypeptide is provided as a composition, such as a pharmaceutical composition, comprising one or more of the polypeptides disclosed herein together with a carrier, such as a pharmaceutically acceptable carrier. In certain examples, the polypeptide is provided as a pharmaceutically acceptable composition comprising one or more polypeptides selected from the group consisting of:
a) a polypeptide comprising, consisting essentially of, or consisting of an amino acid sequence corresponding to residues 1 to 188 of mammalian Decorin;
b) a polypeptide comprising, consisting essentially of, or consisting of an amino acid sequence corresponding to residues 31 to 188 of mammalian Decorin;
c) an N-terminal fragment of mammalian Decorin comprising, consisting essentially of, or consisting of at least 10 contiguous amino acids corresponding to any amino acid sequence within residues 1 to 188 of mammalian Decorin;
d) an N-terminal fragment of mammalian Decorin comprising, consisting essentially of, or consisting of at least 10 contiguous amino acids corresponding to any amino acid sequence within residues 31 to 188 of mammalian Decorin;
e) a polypeptide comprising, consisting essentially of, or consisting of an amino acid sequence corresponding to residues 1 to 177 of mammalian Decorin;
f) a polypeptide comprising, consisting essentially of, or consisting of an amino acid sequence corresponding to residues 31 to 177 of mammalian Decorin;
g) an N-terminal fragment of mammalian Decorin comprising, consisting essentially of, or consisting of at least 10 contiguous amino acids corresponding to any amino acid sequence within residues 1 to 177 of mammalian Decorin;
h) an N-terminal fragment of mammalian Decorin comprising, consisting essentially of, or consisting of at least 10 contiguous amino acids corresponding to any amino acid sequence within residues 31 to 177 of mammalian Decorin;
i) a polypeptide comprising, consisting essentially of, or consisting of an amino acid sequence corresponding to residues 1 to 170 of mammalian Decorin;
j) a polypeptide comprising, consisting essentially of, or consisting of an amino acid sequence corresponding to residues 31 to 170 of mammalian Decorin;
k) a polypeptide comprising, consisting essentially of, or consisting of an amino acid sequence corresponding to residues 31 to 169 of human Decorin;
l) an N-terminal fragment of mammalian Decorin comprising, consisting essentially of, or consisting of at least 10 contiguous amino acids corresponding to any amino acid sequence within residues 1 to 170 of mammalian Decorin;
m) an N-terminal fragment of mammalian Decorin comprising, consisting essentially of, or consisting of at least 10 contiguous amino acids corresponding to any amino acid sequence within residues 31 to 170 of mammalian Decorin;
n) a polypeptide comprising, consisting essentially of, or consisting of the amino acid sequence depicted in any one of Sequence ID No.s: 1 to 13;
o) a polypeptide comprising, consisting essentially of, or consisting of at least about 10 contiguous amino acids from any one of a) to n) above;
p) a polypeptide comprising or consisting of at least about 10 contiguous amino acids from any one of a) to n) above, wherein said polypeptide comprises a motif or region capable of interacting with or recruiting one or more cells, including one or more stem cells; and
q) a polypeptide comprising or consisting of at least about 10 contiguous amino acids from any one of a) to p) above, wherein said polypeptide comprises a motif or region capable of interacting with or recruiting one or more mesenchymal stem cells;
r) a functional fragment, a functional variant, a peptide analogue or peptidomimetic, or a derivative of any one of a) to q) above; and
s) a polypeptide having at least about 90% amino acid identity to any one of a) to r) above.
t) any combination of any two or more of a) to s) above.

In one example, the composition comprises a pharmaceutically acceptable carrier.

In a further aspect, the present invention relates to a method of mediating a biological effect in a subject suffering from or susceptible to tissue ischemia and/or a disease or condition associated with tissue ischemia, the method comprising administering to the subject an effective amount of a polypeptide selected from the group comprising:
a) a polypeptide comprising, consisting essentially of, or consisting of an amino acid sequence corresponding to residues 1 to 188 of mammalian Decorin;
b) a polypeptide comprising, consisting essentially of, or consisting of an amino acid sequence corresponding to residues 31 to 188 of mammalian Decorin;
c) an N-terminal fragment of mammalian Decorin comprising, consisting essentially of, or consisting of at least 10 contiguous amino acids corresponding to any amino acid sequence within residues 1 to 188 of mammalian Decorin;
d) an N-terminal fragment of mammalian Decorin comprising, consisting essentially of, or consisting of at least 10 contiguous amino acids corresponding to any amino acid sequence within residues 31 to 188 of mammalian Decorin;
e) a polypeptide comprising, consisting essentially of, or consisting of an amino acid sequence corresponding to residues 1 to 177 of mammalian Decorin;
f) a polypeptide comprising, consisting essentially of, or consisting of an amino acid sequence corresponding to residues 31 to 177 of mammalian Decorin;
g) an N-terminal fragment of mammalian Decorin comprising, consisting essentially of, or consisting of at least 10 contiguous amino acids corresponding to any amino acid sequence within residues 1 to 177 of mammalian Decorin;
h) an N-terminal fragment of mammalian Decorin comprising, consisting essentially of, or consisting of at least 10 contiguous amino acids corresponding to any amino acid sequence within residues 31 to 177 of mammalian Decorin;
i) a polypeptide comprising, consisting essentially of, or consisting of an amino acid sequence corresponding to residues 1 to 170 of mammalian Decorin;
j) a polypeptide comprising, consisting essentially of, or consisting of an amino acid sequence corresponding to residues 31 to 170 of mammalian Decorin;
k) a polypeptide comprising, consisting essentially of, or consisting of an amino acid sequence corresponding to residues 31 to 169 of human Decorin;
l) an N-terminal fragment of mammalian Decorin comprising, consisting essentially of, or consisting of at least 10 contiguous amino acids corresponding to any amino acid sequence within residues 1 to 170 of mammalian Decorin;
m) an N-terminal fragment of mammalian Decorin comprising, consisting essentially of, or consisting of at least 10 contiguous amino acids corresponding to any amino acid sequence within residues 31 to 170 of mammalian Decorin;
n) a polypeptide comprising, consisting essentially of, or consisting of the amino acid sequence depicted in any one of Sequence ID No.s: 1 to 13;
o) a polypeptide comprising, consisting essentially of, or consisting of at least about 10 contiguous amino acids from any one of a) to n) above;
p) a polypeptide comprising or consisting of at least about 10 contiguous amino acids from any one of a) to o) above, wherein said polypeptide comprises a motif or region capable of interacting with or recruiting one or more cells, including one or more stem cells; and
q) a polypeptide comprising or consisting of at least about 10 contiguous amino acids from any one of a) to p) above, wherein said polypeptide comprises a motif or region capable of interacting with or recruiting one or more mesenchymal stem cells;
r) a functional fragment, a functional variant, a peptide analogue or peptidomimetic, or a derivative of any one of a) to q) above; and
s) a polypeptide having at least about 90% amino acid identity to any one of a) to r) above.
t) any combination of any two or more of a) to s) above.

In one example, the biological effect is mediated in vivo in a subject in need thereof, for example by administration of one or more of the polypeptides to the subject suffering from or susceptible to tissue ischemia and/or a disease or condition associated with tissue ischemia. In one example, the effective amount is a therapeutically effective amount.

In another example, the biological effect is mediated ex vivo, for example, in vitro, for example by contacting a biological sample from the subject with one or more of the polypeptides. For example, the biological effect is mediated in vitro by contacting one or more cells, one or more tissues, or one or more organs from the subject suffering from or susceptible to a disease or condition associated with tissue ischemia with the one or more polypeptides.

In another aspect, the invention relates to a method of modulating tissue repair in a subject suffering from or susceptible to tissue ischemia and/or a disease or condition associated with tissue ischemia, the method comprising administering to the subject a therapeutically effective amount of a polypeptide as described herein.

In certain examples, the therapeutically effective amount is sufficient to recruit one or more stem cells, for example, to the site of administration, or to the site at which the administered polypeptide is localised.

In still another aspect, the invention relates to a method of modulating stem cell recruitment or a related process in a subject suffering from or susceptible to tissue ischemia and/or a disease or condition associated with tissue ischemia, the method comprising administering to the subject a therapeutically effective amount of a polypeptide as described herein.

In various examples, the related process is angiogenesis, hematopoiesis, protein expression, induction, or deposition, tissue remodelling, repair, or regeneration, cellular proliferation, cellular differentiation including stem cell differentiation, cellular regulation, apoptosis, modulation of one or more immune responses, modulation of tumourigenesis, chemotaxis, or cell recruitment.

In various examples, the methods comprise administering to the subject a therapeutically effective amount of a pharmaceutically acceptable composition as described herein. In one example, the administration is parenteral, for example, intravenous administration, or by a non-intravenous parenteral administration route, such as intramuscular, intradermal, or subcutaneous injection. In another example, the administration is by inhalation, instillation, or insufflation, for example by nasal inhalation, nasal instillation, or nasal insufflation. In one example, administration is via nebulisation. In another aspect, the invention relates to a MayDay polypeptide as contemplated herein for treating tissue ischemia and/or a disease or condition associated with tissue ischemia in a subject in need thereof.

In another aspect, the invention relates to a MayDay polypeptide as contemplated herein for treating myocardial infarction, heart failure, and/or cardiomyopathy in a subject in need thereof.

In another aspect, the invention relates to a MayDay polypeptide as contemplated herein for mediating a biological effect in a subject, for modulating stem cell recruitment or a related process in a subject, or for modulating tissue repair in a subject, wherein the subject is suffering from or susceptible to tissue ischemia and/or a disease or condition associated with tissue ischemia.

In another aspect, the invention relates to the use of a MayDay polypeptide as contemplated herein in the preparation of a medicament for the treatment of tissue ischemia and/or a disease or condition associated with tissue ischemia.

In another aspect, the invention relates to the use of a MayDay polypeptide as contemplated herein in the preparation of a medicament for the treatment of myocardial infarction, heart failure, and/or cardiomyopathy in a subject.

In another aspect, the invention relates to the use of a MayDay polypeptide as contemplated herein in the preparation of a medicament for mediating a biological effect in a subject, for modulating stem cell recruitment or a related process in a subject, or for modulating tissue repair in a subject, wherein the subject is suffering from or susceptible to tissue ischemia and/or a disease or condition associated with tissue ischemia.

In one example, the medicament is formulated for parenteral administration. For example, the medicament is formulated for intraveneous administration, or is formulated for non-intraveneous parenteral administration route, such as intramuscular, intradermal, or subcutaneous administration. In another example, the medicament is formulated for administration by inhalation, instillation, or insufflation, for example by nasal inhalation, nasal instillation, or nasal insufflation. In one example, the medicament is formulated for administration by nebulisation.

In various examples, the polypeptide described herein is administered to the subject at a dosage of from about 10 ng/kg to about 20 mg/kg. For example, the polypeptide described herein is administered at a dosage of from about 10 ng/kg to about 10 mg/kg, or from about 50 ng/kg to about 5 mg/kg, 100 ng/kg to about 5 mg/kg, or from about 1 mg/kg to about 5 mg/kg. In various examples, the polypeptide described herein is administered to the subject at a dosage consistent with the dosages exemplified herein in the Examples.

Other aims, aspects, features and advantages of the present invention will become apparent from the following description. It should be understood, however, that the detailed description and the specific examples, while indicating preferred examples of the invention, are given by way of illustration only, since various changes and modifications within the spirit and scope of the invention will become apparent to those skilled in the art from this detailed description.

### BRIEF DESCRIPTION OF THE FIGURES

FIG. 1 presents data from flow cytometry analysis of stem cells markers CD29 (FIG. 1A), CD105 (FIG. 1B), and SCA1 (FIG. 1C) in muscle tissue post intramuscular injection of controls and test articles, as described herein in Example 1. For each marker, counts were analysed at 72 h. The average counts for each marker were expressed and error bars indicate the SD of each group (n=7), statistical significance was calculated via 2 way ANOVA compared with vehicle control, where: ‘*’p≤0.05; ‘**’p≤0.01; ‘***’p≤0.001; ‘***’p≤0.0001.

FIG. 2 presents the results of an immunohistochemical analysis of muscle biopsies as described herein in Example 1. Immunohistochemistry was carried out at three time points for SCA1+ cells. Quantification of SCA1+ cells per field of view are shown in FIG. 2A and representative images from each group at each time point are shown in FIG. 2B. Data presented in FIG. 2A is expressed as average counts per high powered field of view (HPFV) with error bars indicating the SD of each group (n=7), statistical significance was calculated via 2 way ANOVA compared with vehicle control, where: ‘*’p≤0.05; ‘***’p≤0.0001.

FIG. 3 is two graphs presenting the results of echocardiography in a murine model of myocardial infarction (MI) as described herein in Example 2. End-systolic volume (ESV, FIG. 3A) and end-diastolic volume (EDV, FIG. 3B) were measured at 4 weeks and 8 weeks after MI in untreated MI animals (MD (−)), and MI animals to which a representative MayDay polypeptide had been administered (MD (+)). ESV and EDV of control animals which did not undergo MI (Control) was also measured at the 4 week timepoint.

### DETAILED DESCRIPTION

The present invention broadly relates to methods for treating or preventing tissue ischemia and/or a disease or condition associated with tissue ischemia in a subject in need thereof. The methods comprise the administration of a MayDay polypeptide as described herein.

Without wishing to be bound by any theory, the applicants have determined that representative MayDay polypeptides as contemplated herein are bioactive, and are capable of eliciting one or more biological responses, such as one or more biological responses in a subject to which they are administered. In certain examples, and again without wishing to be bound by any theory, the biological response is useful in the treatment or prevention of tissue ischemia, including tissue ischemia associated with a disease or condition. Representative diseases or conditions associated with tissue ischemia include, but are not limited to, ischemic-reperfusion injury in an organ for transplantation, spinal cord injury, myocardial infarction, heart failure, acute kidney injury, renal failure, ischemic stroke, hemorrhagic stroke, acute tubular necrosis, compartment syndrome, coronary artery disease, intestinal ischemia, peripheral artery disease, transient ischemic attack, ischemia due to atherosclerosis, chronic kidney disease, pancreatic ischemia, critical limb ischemia, chronic wounds, cirrhosis, liver failure, liver ischemia, vascular dementia, cutaneous ischemia, ischemia due to thrombosis, ischemia due to embolism, ischemia due to trauma, ischemia due to aneurysm, and traumatic brain injury.

In certain examples, the one or more biological responses include one or more of cellular activation, cellular proliferation, cellular chemotaxis and/or recruitment, cellular differentiation, cellular regulation including cell cycle regulation, and apoptosis. In particular, the one or more biological responses include one or more of stem cell activation, stem cell proliferation, stem cell chemotaxis and/or recruitment, stem cell differentiation, stem cell regulation, and stem cell apoptosis.

In certain examples, the one or more biological responses include one or more of eliciting or modulating an immune response, such as but not limited to upregulation or downregulation of cytokine expression, and upregulation or downregulation of immune cell (e.g. neutrophils, macrophages) activation, proliferation, maturation, or recruitment.

In certain examples, the one or more biological responses include one or more of angiogenesis and/or the modulation of angiogenesis, hematopoiesis and/or the modulation of hematopoiesis, tissue remodelling and/or modulation of tissue repair, wound healing and/or regeneration, the modulation of tissue microenvironments, and modulation of tumourigenesis.

Various aspects of the invention are described in further detail in the following subsections. Unless otherwise defined, all technical and scientific terms used herein have the same meaning as is commonly understood by one of ordinary skill in the art to which this invention belongs. In case of conflict, the present specification, including definitions, will control. Although methods and materials similar or equivalent to those described herein can be used in the practice of the invention, examples of suitable methods and materials are described below. The materials, methods, and examples described herein are illustrative only and are not intended to be limiting.

### Selected Definitions

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7). These are only examples of what is specifically intended and all possible combinations of numerical values between the lowest value and the highest value enumerated are to be considered to be expressly stated in this application in a similar manner.

Those skilled in the art will appreciate the meaning of various terms of degree used herein. For example, as used herein in the context of referring to an amount (e.g., “about 9%”), the term “about” represents an amount close to and including the stated amount that still performs a desired function or achieves a desired result, e.g. “about 9%” can include 9% and amounts close to 9% that still perform a desired function or achieve a desired result. For example, the term “about” can refer to an amount that is within less than 10% of, within less than 5% of, within less than 1% of, within less than 0.1% of, or within less than 0.01% of the stated amount. It is also intended that where the term “about” is used, for example with reference to a figure, concentration, amount, integer or value, the exact figure, concentration, amount, integer or value is also specifically contemplated.

As used herein, the term “and/or” can mean “and” or “or”.

The terms “comprise”, “comprises”, and “comprising” as used in this specification and claims are not to be interpreted in an exclusive or exhaustive sense, and mean “consisting at least in part of”. When interpreting each statement in this specification that includes the term “comprise”, “comprises”, or “comprising”, features other than that or those prefaced by the term may also be present. Related terms such as “including”, “include” and “includes” are to be interpreted in the same manner.

The term “consisting essentially of” when used in this specification refers to the features stated and allows for the presence of other features that do not materially alter the basic characteristics of the features specified.

The term “consisting of” as used herein means the specified materials or steps of the claimed invention, excluding any element, step, or ingredient not specified in the claim.

### MayDay Polypeptides

The invention relates to the therapeutic use of one or more MayDay polypeptides. As contemplated herein, a MayDay polypeptide comprises, consists essentially of, or consists of an amino acid sequence corresponding to that of an N-terminal fragment of mammalian Decorin.

The extracellular matrix protein Decorin (Homo sapiens Gene ID: 1634, NCBI Reference Sequence: NG_011672.2) is involved in collagen fibril assembly and has been reported to have roles in angiogenesis and tumourigenesis.

Table 1 below presents the amino acid sequence of the full length ovine Decorin preproprotein [Sequence ID No.: 1], the amino acid sequence of human Decorin isoform A preproprotein (NCBI accession: NP_598010.1, GI: 19743846 [Sequence ID No.: 5]), the amino acid sequence of murine Decorin preproprotein (NCBI Reference Sequence: NP_001177380.1 [Sequence ID No.: 8]), and the amino acid sequence of the full length bovine Decorin preproprotein [Sequence ID No.: 11]. The amino acid sequence of the recombinant MayDay₃₁₋₁₇₀ polypeptide [Sequence ID No.: 2] exemplified herein in Example 1, and the amino acid sequences of two N-terminal fragments of Decorin each terminating at an MMP12 cleavage site, MayDay₃₁₋₁₇₇ [Sequence ID No.: 3], and MayDay₃₁₋₁₈₈ [Sequence ID No.: 4], respectively, are also depicted in Table 1. The amino acid sequence of a human MayDay₃₁₋₁₆₉ polypeptide [Sequence ID No.: 6], a human MayDay₃₁₋₁₈₇ polypeptide [Sequence ID No.: 7], a murine MayDay₃₁₋₁₆₄ polypeptide [Sequence ID No.: 9], a murine MayDay₃₁₋₁₈₂ polypeptide [Sequence ID No.: 10], a bovine MayDay₃₁₋₁₇₀ polypeptide [Sequence ID No.: 12], and a bovine MayDay₃₁₋₁₈₈ polypeptide [Sequence ID No.: 13], are also presented in Table 1.

TABLE 1
Decorin and MayDay peptides

| | | Sequence |
|---|---|---|
| Polypeptide | Amino acid sequence | ID No.: |
| Ovine decorin | MKATIIFFLV AQVSWAGPFQ QKGLFDFMLE DEASGIGPEE | 1 |
| | RFHEVPELEP MGPVCPFRCQ CHLRVVQCSD LGLEKVPKDL | |
| | PPDTALLDLQ NNKITEIKDG DFKNLKNLHT LILINNKISK | |
| | ISPGAFAPLV KLERLYLSKN QLKELPEKMP KTLQELRVHE | |
| | NEITKVRKSV FNGLNQMIVV ELGTNPLKSS GIENGAFQGM | |
| | KKLSYIRIAD TNITTIPQGL PPSLTELHLD GNKITKVDAA | |
| | SLKGLNNLAK LGLSFNSISA VDNGSLANTP HLRELHLNNN | |
| | KLVKVPGGLA DHKYIQVVYL HNNNISAIGS NDFCPPGYNT | |
| | KKASYSGVSL FSNPVQYWEI QPSTFRCVYV RAAVQLGNYK | |
| Ovine MayDay31-170 | DEASGIGPEE RFHEVPELEP MGPVCPFRCQ CHLRVVQCSD | 2 |
| | LGLEKVPKDL PPDTALLDLQ NNKITEIKDG DFKNLKNLHT | |
| | LILINNKISK ISPGAFAPLV KLERLYLSKN QLKELPEKMP | |
| | KTLQELRVHE NEITKVRKSV | |
| Ovine MayDay31-177 | DEASGIGPEE RFHEVPELEP MGPVCPFRCQ CHLRVVQCSD | 3 |
| | LGLEKVPKDL PPDTALLDLQ NNKITEIKDG DFKNLKNLHT | |
| | LILINNKISK ISPGAFAPLV KLERLYLSKN QLKELPEKMP | |
| | KTLQELRVHE NEITKVRKSV FNGLNQM | |
| Ovine MayDay31-188 | DEASGIGPEE RFHEVPELEP MGPVCPFRCQ CHLRVVQCSD | 4 |
| | LGLEKVPKDL PPDTALLDLQ NNKITEIKDG DFKNLKNLHT | |
| | LILINNKISK ISPGAFAPLV KLERLYLSKN QLKELPEKMP | |
| | KTLQELRVHE NEITKVRKSV FNGLNQMIVV ELGTNPLK | |
| Human decorin | MKATIILLLL AQVSWAGPFQ QRGLFDFMLE DEASGIGPEV | 5 |
| | PDDRDFEPSL GPVCPFRCQC HLRVVQCSDL GLDKVPKDLP | |
| | PDTTLLDLQN NKITEIKDGD FKNLKNLHAL ILVNNKISKV | |
| | SPGAFTPLVK LERLYLSKNQ LKELPEKMPK TLQELRAHEN | |
| | EITKVRKVTF NGLNQMIVIE LGTNPLKSSG IENGAFQGMK | |
| | KLSYIRIADT NITSIPQGLP PSLTELHLDG NKISRVDAAS | |
| | LKGLNNLAKL GLSFNSISAV DNGSLANTPH LRELHLDNNK | |
| | LTRVPGGLAE HKYIQVVYLH NNNISVVGSS DFCPPGHNTK | |
| | KASYSGVSLF SNPVQYWEIQ PSTFRCVYVR SAIQLGNYK | |
| Human MayDay31-169 | DEASGIGPEV PDDRDFEPSL GPVCPFRCQC HLRVVQCSDL | 6 |
| | GLDKVPKDLP PDTTLLDLQN NKITEIKDGD FKNLKNLHAL | |
| | ILVNNKISKV SPGAFTPLVK LERLYLSKNQ LKELPEKMPK | |
| | TLQELRAHEN EITKVRKVT | |
| Human MayDay31-187 | DEASGIGPEV PDDRDFEPSL GPVCPFRCQC HLRVVQCSDL | 7 |
| | GLDKVPKDLP PDTTLLDLQN NKITEIKDGD FKNLKNLHAL | |
| | ILVNNKISKV SPGAFTPLVK LERLYLSKNQ LKELPEKMPK | |
| | TLQELRAHEN EITKVRKVTF NGLNQMIVIE LGTNPLK | |
| Murine decorin | MKATLIFFLL AQVSWAGPFE QRGLFDFMLE DEASGIIPYD | 8 |
| | PDNPLISMCP YRCQCHLRVV QCSDLGLDKV PWDFPPDTTL | |
| | LDLQNNKITE IKEGAFKNLK DLHTLILVNN KISKISPEAF | |
| | KPLVKLERLY LSKNQLKELP EKMPRTLQEL RVHENEITKL | |
| | RKSDFNGLNN VLVIELGGNP LKNSGIENGA FQGLKSLSYI | |
| | RISDTNITAI PQGLPTSLTE VHLDGNKITK VDAPSLKGLI | |
| | NLSKLGLSFN SITVMENGSL ANVPHLRELH LDNNKLLRVP | |
| | AGLAQHKYIQ VVYLHNNNIS AVGQNDFCRA GHPSRKASYS | |
| | AVSLYGNPVR YWEIFPNTFR CVYVRSAIQL GNYK | |
| Murine MayDay31-164 | DEASGIIPYD PDNPLISMCP YRCQCHLRVV QCSDLGLDKV | 9 |
| | PWDFPPDTTL LDLQNNKITE IKEGAFKNLK DLHTLILVNN | |
| | KISKISPEAF KPLVKLERLY LSKNQLKELP EKMPRTLQEL | |
| | RVHENEITKL RKSD | |
| Murine MayDay31-182 | DEASGIIPYD PDNPLISMCP YRCQCHLRVV QCSDLGLDKV | 10 |
| | PWDFPPDTTL LDLQNNKITE IKEGAFKNLK DLHTLILVNN | |
| | KISKISPEAF KPLVKLERLY LSKNQLKELP EKMPRTLQEL | |
| | RVHENEITKL RKSDFNGLNN VLVIELGGNP LK | |
| Bovine decorin | MKATIIFLLV AQVSWAGPFQ QKGLFDFMLE DEASGIGPEE | 11 |
| | HFPEVPEIEP MGPVCPFRCQ CHLRVVQCSD LGLEKVPKDL | |
| | PPDTALLDLQ NNKITEIKDG DFKNLKNLHT LILINNKISK | |
| | ISPGAFAPLV KLERLYLSKN QLKELPEKMP KTLQELRVHE | |
| | NEITKVRKSV FNGLNQMIVV ELGTNPLKSS GIENGAFQGM | |
| | KKLSYIRIAD TNITTIPQGL PPSLTELHLD GNKITKVDAA | |
| | SLKGLNNLAK LGLSFNSISA VDNGSLANTP HLRELHLNNN | |
| | KLVKVPGGLA DHKYIQVVYL HNNNISAIGS NDFCPPGYNT | |
| | KKASYSGVSL FSNPVQYWEI QPSTFRCVYV RAAVQLGNYK | |
| Bovine MayDay31-170 | DEASGIGPEE HFPEVPEIEP MGPVCPFRCQ CHLRVVQCSD | 12 |
| | LGLEKVPKDL PPDTALLDLQ NNKITEIKDG DFKNLKNLHT | |
| | LILINNKISK ISPGAFAPLV KLERLYLSKN QLKELPEKMP | |
| | KTLQELRVHE NEITKVRKSV | |
| Bovine MayDay31-188 | DEASGIGPEE HFPEVPEIEP MGPVCPFRCQ CHLRVVQCSD | 13 |
| | LGLEKVPKDL PPDTALLDLQ NNKITEIKDG DFKNLKNLHT | |
| | LILINNKISK ISPGAFAPLV KLERLYLSKN QLKELPEKMP | |
| | KTLQELRVHE NEITKVRKSV FNGLNQMIVV ELGTNPLK | |

In one example, the MayDay polypeptide is a polypeptide selected from the group comprising:
a) a polypeptide comprising, consisting essentially of, or consisting of an amino acid sequence corresponding to residues 1 to 188 of mammalian Decorin;
b) a polypeptide comprising, consisting essentially of, or consisting of an amino acid sequence corresponding to residues 31 to 188 of mammalian Decorin;
c) an N-terminal fragment of mammalian Decorin comprising, consisting essentially of, or consisting of at least 10 contiguous amino acids corresponding to any amino acid sequence within residues 1 to 188 of mammalian Decorin;
d) an N-terminal fragment of mammalian Decorin comprising, consisting essentially of, or consisting of at least 10 contiguous amino acids corresponding to any amino acid sequence within residues 31 to 188 of mammalian Decorin;
e) a polypeptide comprising, consisting essentially of, or consisting of an amino acid sequence corresponding to residues 1 to 177 of mammalian Decorin;
f) a polypeptide comprising, consisting essentially of, or consisting of an amino acid sequence corresponding to residues 31 to 177 of mammalian Decorin;
g) an N-terminal fragment of mammalian Decorin comprising, consisting essentially of, or consisting of at least 10 contiguous amino acids corresponding to any amino acid sequence within residues 1 to 177 of mammalian Decorin;
h) an N-terminal fragment of mammalian Decorin comprising, consisting essentially of, or consisting of at least 10 contiguous amino acids corresponding to any amino acid sequence within residues 31 to 177 of mammalian Decorin;
i) a polypeptide comprising, consisting essentially of, or consisting of an amino acid sequence corresponding to residues 1 to 170 of mammalian Decorin;
j) a polypeptide comprising, consisting essentially of, or consisting of an amino acid sequence corresponding to residues 31 to 170 of mammalian Decorin;
k) a polypeptide comprising, consisting essentially of, or consisting of an amino acid sequence corresponding to residues 31 to 169 of human Decorin;
l) an N-terminal fragment of mammalian Decorin comprising, consisting essentially of, or consisting of at least 10 contiguous amino acids corresponding to any amino acid sequence within residues 1 to 170 of mammalian Decorin;
m) an N-terminal fragment of mammalian Decorin comprising, consisting essentially of, or consisting of at least 10 contiguous amino acids corresponding to any amino acid sequence within residues 31 to 170 of mammalian Decorin;
n) a polypeptide comprising, consisting essentially of, or consisting of the amino acid sequence depicted in any one of Sequence ID No.s: 1 to 13;
o) a polypeptide comprising, consisting essentially of, or consisting of at least about 10 contiguous amino acids from any one of a) to n) above;
p) a polypeptide comprising or consisting of at least about 10 contiguous amino acids from any one of a) to o) above, wherein said polypeptide comprises a motif or region capable of interacting with or recruiting one or more cells, including one or more stem cells; and
q) a polypeptide comprising or consisting of at least about 10 contiguous amino acids from any one of a) to p) above, wherein said polypeptide comprises a motif or region capable of interacting with or recruiting one or more mesenchymal stem cells;
r) a functional fragment, a functional variant, a peptide analogue or peptidomimetic, or a derivative of any one of a) to q) above; and
s) a polypeptide having at least about 90% amino acid identity to any one of a) to r) above.

Those skilled in the art will recognise, on reading this description, that these polypeptides can be considered representative examples of the MayDay polypeptides contemplated herein. Various therapeutic and prophylactic uses of and for these polypeptides, for example in therapeutic methods involving cellular chemotaxis and recruitment, such as tissue remodelling, modulation of tissue repair and wound healing, the modulation of immune responses, the modulation of angiogenesis, and the modulation of tissue microenvironments, for example, are contemplated.

MayDay polypeptides suitable for use herein include polypeptides having an amino acid sequence identical to that found within a naturally occurring Decorin protein, such as that of an N-terminal fragment of a naturally occurring Decorin protein, and variants or derivatives thereof including polypeptides having one or more amino acid variations from the amino acid sequence of a naturally-occurring Decorin protein.

The term “amino acid” refers to natural amino acids, non-natural amino acids, and amino acid analogues. Unless otherwise indicated, the term “amino acid” includes both D and L stereoisomers if the respective structure allows such stereoisomeric forms.

Natural amino acids include alanine (Ala or A), arginine (Arg or R), asparagine (Asn or N), aspartic acid (Asp or D), cysteine (Cys or C), glutamine (Gin or Q), glutamic acid (Glu or E), glycine (Gly or G), histidine (His or H), isoleucine (He or I), leucine (Leu or L), Lysine (Lys or K), methionine (Met or M), phenylalanine (Phe or F), proline (Pro or P), serine (Ser or S), threonine (Thr or T), tryptophan (Tip or W), tyrosine (Tyr or Y) and valine (Val or V).

Non-natural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethyl glycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), Nalkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analogue” refers to a natural or non-natural amino acid where one or more of the C-terminal carboxy group, the N-terminal amino group and side-chain functional group has been chemically blocked, reversibly or irreversibly, or otherwise modified to another functional group. For example, aspartic acid-(beta-methyl ester) is an amino acid analogue of aspartic acid; N-ethylglycine is an amino acid analogue of glycine; or alanine carboxamide is an amino acid analogue of alanine. Other amino acid analogues include methionine sulfoxide, methionine sulfone, S-(carboxymethyl)-cysteine, S-(carboxymethyl) cysteine sulfoxide and S-(carboxymethyl)-cysteine sulfone.

The term “expression construct” refers to a genetic construct that includes elements that permit transcribing the polynucleotide molecule of interest, and, optionally, translating the transcript into a polypeptide. An expression construct typically comprises in a 5′ to 3′ direction:
(1) a promoter, functional in the host cell into which the construct will be introduced,
(2) the polynucleotide to be expressed, and
(3) a terminator functional in the host cell into which the construct will be introduced.

Expression constructs of the invention are inserted into a replicable vector for cloning or for expression, or are incorporated into the host genome.

The term “vector” as used herein refers to a polynucleotide molecule, usually but not limited to a double stranded DNA, which is amenable to use in molecular biological techniques, for example to modify, manipulate, replicate, amplify, or transport a polynucleotide molecule. In certain examples, a vector is used to transport a polynucleotide molecule, such as but not limited to a genetic construct, for example an expression construct, into a host cell or organism. In certain examples the vector is capable of replication and/or maintenance in more than one host system.

A “fragment” of a polypeptide is a subsequence of the polypeptide, typically one that performs a function that is required for activity, such as enzymatic or binding activity, and/or provides a three dimensional structure of the polypeptide or a part thereof, such as an epitope. It will be appreciated that a fragment of a polypeptide may possess or elicit a different function or functions from that possessed or exhibited by the full-length polypeptide from which it is derived.

As used herein, the term “peptide” refers to a short polymer of amino acids linked together by peptide bonds. While it will be recognised that the names associated with various classes of amino acid polymers (e.g., peptides, proteins, polypeptides, etc.) are somewhat arbitrary, peptides are generally of about 50 amino acids or less in length. A peptide can comprise natural amino acids, non-natural amino acids, amino acid analogues, and/or modified amino acids. A peptide can be a subsequence of naturally occurring protein or a non-natural, including a synthetic, sequence.

As used herein, the term “synthetic peptide” encompasses a peptide having a distinct amino acid sequence from those found in natural peptides and/or proteins. A “synthetic peptide,” as used herein, can be produced or synthesized by any suitable method (e.g., recombinant expression, for example via an expression construct, chemical synthesis, enzymatic synthesis, etc.), and can include any chemical modification to a parent peptide, and may include, but is not limited to such methods as truncations, deletions, cyclization or non-peptidic synthetic or semi-synthetic derivatives that retain the same biological function(s) as the starting peptide. Methods of protein synthesis, such as solid state synthesis, are well known in the art.

The terms “peptide analogue”, “peptide mimetic” or “peptidomimetic” refer to a peptide-like molecule that emulates a sequence derived from a protein or peptide. A peptide analogue, peptide mimetic or peptidomimetic can contain amino acids and/or non-amino acid components. Examples include chemically modified peptides, peptoids (side groups are appended to the nitrogen atom of the peptide backbone, rather than to the α-carbons), β-peptides (amino group bonded to the β carbon rather than the α-carbon), etc. Chemical modification includes one or more modifications at amino acid side groups, α-carbon atoms, terminal amine group, or terminal carboxy group. A chemical modification can be adding chemical moieties, creating new bonds, or removing chemical moieties. Modifications at amino acid side groups include, without limitation, acylation of lysine ε-amino groups, N-alkylation of arginine, histidine, or lysine, alkylation of glutamic or aspartic carboxylic acid groups, lactam formation via cyclization of lysine ε-amino groups with glutamic or aspartic acid side group carboxyl groups, hydrocarbon “stapling” (e.g., to stabilize alpha-helix conformations), and deamidation of glutamine or asparagine. Modifications of the terminal amine group include, without limitation, the desamino, N-lower alkyl, N-di-lower alkyl, constrained alkyls (e.g. branched, cyclic, fused, adamantyl) and N-acyl modifications. Modifications of the terminal carboxy group include, without limitation, the amide, lower alkyl amide, constrained alkyls (e.g. branched, cyclic, fused, adamantyl) alkyl, dialkyl amide, and lower alkyl ester modifications. Lower alkyl is C1-C4 alkyl. Furthermore, one or more side groups, or terminal groups, can be protected by protective groups known to the ordinarily skilled peptide chemist. The a-carbon of an amino acid can be mono- or dimethylated.

It will be appreciated that any one of the proteins or peptides described herein in certain examples comprises one or more non-naturally occurring amino acids, one or more amino acid analogues, or is or comprises a synthetic peptide, synthetic polypeptide, a peptide analogue, or a peptide mimetic. Similarly, it will be appreciated that any one of the proteins or peptides described herein will in certain examples be the starting point for one or more modifications, synthetic methods, or protein engineering methods to develop a peptide analogue having a desired biological activity—for example, a qualitatively similar bioactivity as the parent protein or peptide, but an effect of a quantitatively different magnitude, or indeed a different bioactivity from that elicited by the parent protein or peptide.

The term “fusion polypeptide”, as used herein, refers to a polypeptide comprising two or more amino acid sequences, for example two or more polypeptide domains, fused through respective amino and carboxyl residues by a peptide linkage to form a single continuous polypeptide. It should be understood that the two or more amino acid sequences can either be directly fused or indirectly fused through their respective amino and carboxyl terminii through a linker or spacer or an additional polypeptide.

The term “polypeptide”, as used herein, encompasses amino acid chains of any length but preferably at least 10 amino acids, including full-length proteins, in which amino acid residues are linked by covalent peptide bonds. Polypeptides described herein are purified natural products, or are produced partially or wholly using recombinant or synthetic techniques. The term may refer to a polypeptide, an aggregate of a polypeptide such as a dimer or other multimer, a fusion polypeptide, a polypeptide variant, or derivative thereof.

It will be understood that, for the particular polypeptides and proteins contemplated herein, natural variations can exist between individual organisms, including those of the same species or strain. These variations may be demonstrated by (an) amino acid difference(s) in the overall sequence or by deletions, substitutions, insertions, inversions or additions of (an) amino acid(s) in said sequence. Amino acid substitutions which do not essentially alter biological and immunological activities, are well known. Amino acid replacements between related amino acids or replacements which have occurred frequently in evolution are, inter alia, Ser/Ala, Ser/Gly, Asp/Gly, Asp/Asn, Ile/Val. Other amino acid substitutions include Asp/Glu, Thr/Ser, Ala/Gly, Ala/Thr, Ser/Asn, Ala/Val, Thr/Phe, Ala/Pro, Lys/Arg, Leu/Ile, Leu/Val and Ala/Glu. Based on this information, methods for rapid and sensitive protein comparison and determining the functional similarity between homologous proteins were developed. Such amino acid substitutions and variations having deletions and/or insertions are within the scope of the invention as long as the resulting proteins retain their activity, such as their immune reactivity. This explains why one or more proteins described herein may have identity levels below 100%, while still representing the same protein with the same functional (including, for example, immunological) characteristics. Those variations in the amino acid sequence of a certain protein described herein that still provide a protein capable of mediating one or more functions associated with a protein specifically identified herein are considered as functional equivalents of the proteins identified herein, and as such do not essentially influence the functionality of the protein.

When a protein is used for example for diagnostic or therapeutic purposes, for example for reacting with antibodies, or for mediating a biological effect, for example one or more of the biological functions associated with the native protein or a fragment thereof in vivo, while it can be expedient to do so it is not necessary to use the whole protein. It is also possible to use a polypeptide fragment of that protein (as such or coupled to a carrier or as a component in a fusion polypeptide, for example) or a polypeptide fragment derived from that protein or a related amino acid sequence that is capable of eliciting a desired biological effect, such as an immune response against that protein or of being recognised by an antibody specific to that protein, of mediating a cell-signalling effect, or the like. Such a polypeptide fragment may be referred to with reference to the function it possesses, such as the function it shares with the full-length protein from which it was derived. For example, a polypeptide fragment having an immunological effect may be referred to as an immunogenic fragment, where an “immunogenic fragment” is understood to be a fragment of the full-length protein that retains its capability to induce an immune response in a vertebrate host or be recognised by an antibody specific to the parent protein. Similarly, a polypeptide fragment retaining or possessing one or more biological effects elicited by the full-length protein from which it was derived, or possessing a related or different biological effect, is referred to herein as a “bioactive fragment” or a “bioactive polypeptide fragment”. Likewise, a polypeptide having a biological effect, such as a polypeptide capable of stimulating a biological response in a cell or eliciting a therapeutic effect, may be referred to herein as a “bioactive fragment” or a “bioactive polypeptide fragment”, or grammatical equivalents thereof.

A variety of techniques is available to identify such polypeptide fragments, as well as DNA fragments encoding such fragments. For example, in the case of immunogenic fragments, such fragments may comprise one or more determinants or epitopes. Well-established empirical and in silico methods for the detection of epitopes exist and are well known to those skilled in the art. For example, computer algorithms are able to designate specific protein fragments as the immunologically important epitopes on the basis of their sequential and/or structural agreement with epitopes that are known. The determination of these regions is typically based on a combination of the hydrophilicity criteria and secondary structural features. An immunogenic fragment (or epitope) usually has a minimal length of 6, more commonly 8 amino acids, preferably more than 8, such as 9, 10, 12, 15 or even 20 or more amino acids. The nucleic acid sequences encoding such a fragment therefore have a length of at least 18, more commonly 24 and preferably 27, 30, 36, 45 or even 60 nucleic acids.

Similarly, those skilled in the art will be aware of methods to identify bioactive fragments using various assays targeted at identifying or detecting a particular biological response. Representative methods suitable for use in the identification or detection of bioactive fragments contemplated herein are presented below, including in the Examples.

The term “variant” with reference to polypeptides encompasses naturally occurring, recombinantly, and synthetically produced polypeptides, including those comprising one or more non-natural amino acids, one or more amino acid analogues, peptide analogues, and peptide mimetics. Variant polypeptide sequences preferably exhibit at least 50%, more preferably at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least %, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a sequences of the present invention. Identity is found over a comparison window of at least 20 amino acid positions, preferably at least 50 amino acid positions, at least 100 amino acid positions, or over the entire length of a polypeptide of the invention.

Polypeptide sequence identity can be determined in the following manner. The subject polypeptide sequence is compared to a candidate polypeptide sequence using BLASTP (from the BLAST suite of programs, version 2.2.10 [October 2004]) in bl2seq, which is publicly available from NCBI (ftp://ftp.ncbi.nih.gov/blast/). The default parameters of bl2seq are utilized except that filtering of low complexity regions should be turned off.

Polypeptide sequence identity may also be calculated over the entire length of the overlap between a candidate and subject polynucleotide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

Polypeptide variants contemplated herein also encompass those which exhibit a similarity to one or more of the specifically identified sequences that are likely to preserve the functional equivalence of one or more of those sequences and which could not reasonably be expected to have occurred by random chance. Such sequence similarity with respect to polypeptides can be determined using the publicly available bl2seq program from the BLAST suite of programs (version 2.2.10 [October 2004]) from NCBI (ftp://ftp.ncbi.nih.gov/blast/). The similarity of polypeptide sequences can be examined using the following unix command line parameters:
bl2seq -i peptideseq1 -j peptideseq2-F F -p blastp

Variant polypeptide sequences preferably exhibit an E value of less than 1×10⁻¹⁰, more preferably less than 1×10⁻²⁰, less than 1×10⁻³⁰, less than 1×10⁻⁴⁰, less than 1×10⁻⁵⁰, less than 1×10⁻⁶⁰, less than 1×10⁻⁷⁰, less than 1×10⁻⁸⁰, less than 1×10⁻⁹⁰, less than 1×10⁻¹⁰⁰, less than 1×10⁻¹¹⁰, less than 1×10⁻¹²⁰ or less than 1×10⁻¹²³ when compared with any one of the specifically identified sequences.

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Conservative substitutions of one or several amino acids of a described polypeptide sequence without significantly altering its biological activity are also included in the invention. A skilled artisan will be aware of methods for making phenotypically silent amino acid substitutions (see, e.g., Bowie et al., 1990, Science 247, 1306).

A MayDay polypeptide variant contemplated herein also encompasses that which is produced from the nucleic acid encoding a polypeptide, but differs from the wild-type polypeptide in that it is processed differently such that it has an altered amino acid sequence. For example, in one example a MayDay variant is produced by an alternative splicing pattern of the primary RNA transcript to that which produces the or a wild-type polypeptide.

### Therapeutic Methods and Compositions

Therapeutic methods reliant on the MayDay polypeptides described herein are particularly contemplated, for example, in the treatment or prevention of tissue ischemia, such as tissue ischemia associated with a disease or condition in a subject in need thereof.

As used herein, a disease or condition includes diseases, disorders, pathologies, or conditions in a subject, and includes any symptoms of or sequelae associated with said disease, condition, disorder, or pathology.

A “subject” as used herein is an animal, usually a mammal, including a mammalian companion animal or a human. Representative companion animals include feline, equine, and canine. Representative agricultural animals include bovine, ovine, caprine, cervine, and porcine.

It will be appreciated that the various methods of therapy contemplated herein will typically embody the administration of an effective amount of a MayDay polypeptide.

An “effective amount” is an amount sufficient to elicit one or more beneficial or desired results including one or more clinical results. An effective amount can be administered in one or more administrations by various routes of administration. The effective amount will vary depending on, among other factors, the disease or condition indicated, the severity of the disease or condition, the age and relative health of the subject, the potency of the agent administered, the mode of administration and the treatment desired. A person skilled in the art will be able to determine appropriate dosages having regard to these or any other relevant factors.

The treatment and/or prevention of tissue ischemia, such as tissue ischemia associated with particular diseases or conditions (including as discussed above disorders, pathologies or symptoms or sequelae thereof) is contemplated. Tissue ischemia is a pathology that may manifest in acute or chronic forms, and has a number of symptoms including fatigue, weakness, trouble breathing or shortness of breath, discomfort or pain, difficulty moving, dizziness, difficulty speaking or slurred speech, bloating, diarrhea, and nausea.

Furthermore, tissue ischemia is associated with a number of diseases or conditions of significant clinical consequence. Particularly contemplated examples of diseases or conditions associated with tissue ischemia include ischemic-reperfusion injury in an organ for transplantation, spinal cord injury, myocardial infarction, heart failure, acute kidney injury, renal failure, ischemic stroke, hemorrhagic stroke, acute tubular necrosis, compartment syndrome, coronary artery disease, intestinal ischemia, peripheral artery disease, transient ischemic attack, ischemia due to atherosclerosis, chronic kidney disease, pancreatic ischemia, critical limb ischemia, chronic wounds, cirrhosis, liver failure, liver ischemia, vascular dementia, cutaneous ischemia, ischemia due to thrombosis, ischemia due to embolism, ischemia due to trauma, ischemia due to aneurysm, and traumatic brain injury.

In certain examples, the disease or condition is selected from the group consisting of: myocardial infarction, heart failure, and stroke.

In certain further examples the disease or condition is selected from the group consisting of: acute kidney injury, acute kidney disease, renal failure, and chronic wounds.

The term “treatment”, and related terms such as “treating” and “treat”, as used herein relates generally to treatment, of a human or a non-human subject, in which some desired therapeutic effect is achieved. The therapeutic effect may, for example, be inhibition, reduction, amelioration, halt, cure, mitigation, delay of onset or of progression, or prevention of a disease or condition in a subject, or any activity that otherwise affects the structure or any function of the body of the subject, and includes a treatment relieving, reducing or alleviating at least one symptom or sequelae in a subject or effecting a delay of onset or of progression of a disease or condition or symptom or sequelae thereof. For example, treatment can be the diminishment of one or several symptoms or sequelae of a disorder or complete eradication of a disorder. Within the meaning of the present disclosure, the term “treat” also denotes to arrest, delay the onset (i.e., the period prior to clinical manifestation of a disease) and/or reduce the risk of developing or worsening a disease. “Treatment” can also mean prolonging survival as compared to expected survival if not receiving treatment, for example, an increase in overall survival (OS) compared to a subject not receiving treatment as described herein, and/or an increase in progression-free survival (PFS) compared to a subject not receiving treatment as described herein. The term “treating” can also mean an improvement in the condition of a subject having a disease or condition, e.g., one or more of a decrease in a marker for the disease or condition in a subject, a decrease or no substantial increase in the rate of disease progression in a subject, and an improvement in one or more physiological or metabolic responses or metrics in a subject (e.g., as compared to the one or more metric(s) in a subject having a similar disease or condition receiving no treatment or a different treatment, or as compared to the one or more metric(s) in the same subject prior to treatment). Prophylactic treatments, including those in which treatment is administered before one or more indicia or symptoms of a disease or condition manifest, are also contemplated.

The term “stem cells” as used herein refers to cells capable of self-renewal without differentiation and capability to differentiate into other cell types. The term “stem cells” will, as the context confers, encompass totipotent, pluripotent, and multipotent stem cells.

Generally, and without wishing to be bound by any theory, the methods contemplated herein are in certain examples directed to the modulation of one or more endogenous stem cells in a subject.

Nevertheless, in certain examples ex vivo treatment methods and/or a combination of ex vivo and in vivo treatment methods are contemplated, for example when one or more stem cells that have been cultured in vitro following isolation from a tissue source containing stem cells are employed. In one example, the stem cells are mesenchymal stem cells.

Stem cells are able to differentiate into other cell types and also have capacity to self-renew without differentiation. By supplying various differentiated functional cells as required, stem cells are critical for the formation of new tissues and also the repair of damaged or diseased tissues [Li, L., & Xie, T. (2005). Stem cell niche: structure and function. Annu. Rev. Cell Dev. Biol., 21, 605-631.]. In addition to this progenitor function, stem cells also exhibit their own functionality to promote tissue regeneration and repair, for example via secretion of bioactive factors and through modulating the behaviour of other cell types [Duscher, D., Barrera, J., Wong, V. W., Maan, Z. N., Whittam, A. J., Januszyk, M., & Gurtner, G. C. (2016). Stem cells in wound healing: the future of regenerative medicine? A mini-review. Gerontology, 62 (2), 216-225.]. As part of normal tissue growth and repair, local endogenous stem cells carry out these functions but in circumstances of extensive tissue damage or when coinciding disease factors are present the normal endogenous stem cell population may not be sufficient [Kanji, S., & Das, H. (2017). Advances of stem cell therapeutics in cutaneous wound healing and regeneration. Mediators of inflammation, 2017.].

Stem cell therapy is recognised as having great potential in regenerative medicine, with the administration of additional stem cells demonstrating improved outcomes in a range of injury and disease states including dermal wounds [Falanga, V., Iwamoto, S., Chartier, M., Yufit, T., Butmarc, J., Kouttab, N., & Carson, P. (2007). Autologous bone marrow-derived cultured mesenchymal stem cells delivered in a fibrin spray accelerate healing in murine and human cutaneous wounds. Tissue engineering, 13 (6), 1299-1312.], nervous system injury [di Summa, P. G., Kingham, P. J., Raffoul, W., Wiberg, M., Terenghi, G., & Kalbermatten, D. F. (2010). Adipose-derived stem cells enhance peripheral nerve regeneration. Journal of Plastic, Reconstructive & Aesthetic Surgery, 63 (9), 1544-1552.] and myocardial infarction [Berry, M. F., Engler, A. J., Woo, Y. J., Pirolli, T. J., Bish, L. T., Jayasankar, V., & Sweeney, H. L. (2006). Mesenchymal stem cell injection after myocardial infarction improves myocardial compliance. American Journal of Physiology-Heart and Circulatory Physiology, 290 (6), H2196-H2203.]. Bone marrow-derived stem cells such as MSCs are also capable of differentiating into cells that are required for tissue repair at the site of injury, including pericytes, endothelial cells and keratinocytes. MSCs, along with fibroblasts, fibrocytes and pericytes can differentiate into myofibroblast progenitors that are stimulated by cell matrix interactions, matrix stiffness and mechanical stress to become myofibroblasts—the primary matrix producing cells in wound healing. MSCs also support hematopoiesis by maintaining the hematopoietic stem cell niche.

For example, in the adult bone marrow, mesenchymal stem cells (MSCs) are a pool of regenerative cells that are capable of self-renewal and which play an important role in tissue repair. MSCs release cytokines that modulate the inflammatory response and trophic factors that promote healing processes such as cell recruitment, angiogenesis and collagen synthesis.

Recruitment of cells expressing the stem cell marker SCA1 to the site of tissue injury is exemplified herein in Example 1.

As well as being able to differentiate into the type of cells required at the site of injury, MSCs have important roles in the regulation of other cells, including immune cells and other wound healing cells. MSCs release paracrine signals to recruit keratinocytes, dermal fibroblasts and other nearby stem cells. MSCs increase the rate of fibroblast migration, proliferation and collagen synthesis as well as endothelial cell tube formation. MSCs also secrete important wound healing growth factors such as keratinocyte growth factor (KGF), VEGF and PDGF. Finally, during remodelling, MSCs regulate the expression of MMPs and collagen deposition.

Without wishing to be bound by any theory, the applicants believe that the administration of a MayDay polypeptide is effective to promote tissue repair or regeneration, and/or to prevent tissue damage, and is effective to treat or prevent tissue ischemia, whether via its activity in modulating and/or recruiting one or more stem cells, via promotion of tissue repair or prevention of damage, or via modulation of other biological processes such as one or more immunological responses. Such biological processes may include but are not limited to angiogenesis, vascularogenesis, tissue remodelling, and concentration and/or recruitment and/or activation of one or more populations of cells, such as circulating stem cells or immune cells.

During the inflammatory stage of tissue healing, MSCs regulate the immune response by blocking T-cell proliferation, reducing the inflammatory markers TNF-α, IL-1 and IL-6, as well as producing anti-inflammatory markers such as IL-10 and IL-4. The immunomodulatory and anti-inflammatory activity of MSCs has been shown to be beneficial in abhorrent inflammation associated with various diseases and conditions. Tissue ischemia is typically associated with a substantial release of dysregulated inflammatory cytokines which can lead to further tissue injury. MSCs have been shown to exert broad anti-inflammatory effects in numerous cases of abhorrent tissue inflammation. Specifically, systemic administration of exogenous MSCs has been shown to improve clinical outcomes including a significant reduction in myocardial infarction and stroke, and a reduction in major adverse cardiac events (cardiac death, myocardial infarction, stroke), among ischemic cardiomyopathy patients (DREAM-HF trial, reviewed in Bolli et al., 2022). Without wishing to be bound by any theory, the applicants expect the immunomodulatory effects of MSCs whereby T-cell proliferation is blocked, inflammatory markers are down regulated, and anti-inflammatory markers are elevated, contributes to these clinical outcomes.

It will be appreciated by those skilled in the art that various immune cells populations (for example, macrophages, neutrophils, B-cells, and dendritic cells) will be involved in certain immune responses, including an inflammatory response such as those observed in diseases or conditions associated with tissue ischemia. In certain such disease, other cell types, such as endothelial cells or stem cells (e.g. mesenchymal stem cells and progenitor cells) are, without wishing to be bound by any theory, expected to also play a role in the onset, progression, and in some cases resolution of a disease or condition associated with tissue ischemia.

Notwithstanding advances in cell administration techniques, ex vivo methods can be inefficient and invasive, requiring donor tissue harvesting, extraction, enrichment and re-administration of the isolated cell populations. Therapeutic agents capable of eliciting one or more therapeutic effects, such as a biological response such as inducing localized recruitment of endogenous stem cells are of considerable potential utility in the treatment of conditions which may benefit from the activity of stem cells.

Stem cell therapies, such as therapies utilising MSCs will in certain examples involve the extraction from a donor of a stem cell population, followed by ex vivo culturing and finally administration. The stem cells can be sourced from either an autologous or an allogeneic source. Autologous MSCs are derived from the subject to whom the treatment is to be administered-effectively, the subject acts as their own donor. Allogeneic MSCs are from a person other than the patient, either a matched related or unrelated donor. Therapies based on the administration of either allogeneic or autologous MSCs require extraction from a donor, prior to ex vivo culturing and eventual administration. Such therapies will sometimes require ongoing cotherapies, such as immunosuppression. These cell-based therapies are costly, complex, challenging from a quality control perspective, and may require ongoing patient monitoring.

In contrast, examples of the methods contemplated herein envisage the involvement of endogenous cells (such as endogenous MSCs)—cells already present within the subject, or able to be induced within the subject. These therapies therefore avoid the need for cell extraction or culturing, and as the endogenous cells are immunologically ‘self’, no immunosuppression is required. Pharmacological activation of endogenous stem cells, such as stem cell activation and/or mobilization such as from the blood or a tissue-specific niche, for example using a MayDay polypeptide as herein contemplated, is thus capable of providing the therapeutic benefits of therapies utilising autologous or allogeneic MSCs without the need for donors, cell extraction, ex vivo culturing, and transplantation.

Administration of MayDay polypeptides has been shown to increase the tissue concentration of stem cells [Dempsey, S. G., C. H. Miller, J. Schueler, R. W. F. Veale, D. J. Day and B. C. H. May (2020). “A novel chemotactic factor derived from the extracellular matrix protein decorin recruits mesenchymal stromal cells in vitro and in vivo.” PLoS One 15 (7): e0235784]. MayDay polypeptides have also been shown to recruit endogenous stem cells. Therapeutic administration of MayDay polypeptides may cause a therapeutic increase in stem cells in the appropriate disease-associated tissues, thereby negating the need for ex vivo allogeneic or autologous stem cells.

Again without wishing to be bound by any theory, the applicants believe that the local recruitment of stem cells is broadly beneficial for soft tissue repair, and for the treatment, prevention, management of, or intervention in, tissue ischemia and/or a disease or condition associated with tissue ischemia.

Without wishing to be bound by any theory, the methods contemplated herein are in certain examples directed to the modulation of one or more cellular responses in the subject to which a MayDay polypeptide as contemplated herein is administered.

In one example, the one or more cellular responses is the upregulation or downregulation of cytokine expression, or the modulation of the responsiveness of one or more cells to one or more cytokines.

In one example, the one or more cellular responses is the upregulation or downregulation of immune cell activation, or the modulation of the responsiveness of one or more immune cells to upregulation or downregulation.

In one example, the one or more cellular response is stem cell activation or recruitment, such as mesenchymal stem cell activation or recruitment. In one example, the one or more cellular response is the recruitment or activation of cells expressing a cell surface marker selected from the groups consisting of CD29, CD105, and SCA1.

In various examples, a method of treating or preventing tissue ischemia in a subject in need thereof comprises administration of a MayDay polypeptide as herein contemplated in an amount effective to increase the number, activity, or recruitment of one or more stem cells to a tissue of interest, such as an ischemic tissue.

In various examples, a method of treating or preventing tissue ischemia in a subject in need thereof comprises administration of a MayDay polypeptide as herein contemplated in an amount effective to reduce the amount or concentration of one or more pro-inflammatory cytokines.

In various examples, a method of treating or preventing a disease or condition associated with tissue ischemia in a subject in need thereof comprises administration of a MayDay polypeptide as herein contemplated in an amount effective to reduce the amount or concentration of one or more pro-inflammatory cytokines.

In various examples, a method of treating or preventing tissue ischemia in a subject in need thereof comprises administration of a MayDay polypeptide as herein contemplated in an amount effective to reduce the number, activity, or recruitment, for example recruitment to the ischemic tissue, of one or more immune cells. For example, the method comprises administration of a MayDay polypeptide in an amount effective to reduce the number, activity, or recruitment, for example recruitment to the ischemic tissue, of one or more immune cells selected from the group consisting of macrophages, neutrophils, B-cells, and dendritic cells. In a particularly contemplated example, the method comprises administration of a MayDay polypeptide in an amount effective to reduce the recruitment of neutrophils to muscle tissue. In another particularly contemplated example, the method comprises administration of a MayDay polypeptide in an amount effective to reduce the number or activity of neutrophils in muscle tissue.

In various examples, a method of treating or preventing a disease or condition associated with tissue ischemia in a subject in need thereof comprises administration of a MayDay polypeptide as herein contemplated in an amount effective to reduce the number, activity, or recruitment, for example recruitment to the ischemic tissue, of one or more immune cells. For example, the method comprises administration of a MayDay polypeptide in an amount effective to reduce the number, activity, or recruitment, for example recruitment to the ischemic tissue, of one or more immune cells selected from the group consisting of macrophages, neutrophils, B-cells, and dendritic cells. In a particularly contemplated example, the method comprises administration of a MayDay polypeptide in an amount effective to reduce the recruitment of neutrophils to muscle tissue. In another particularly contemplated example, the method comprises administration of a MayDay polypeptide in an amount effective to reduce the number or activity of neutrophils to muscle tissue.

In particularly contemplated examples, the reduction is a statistically significant reduction, such as a statistically significant reduction compared to a vehicle control.

### Compositions and Medicaments

In one aspect the invention relates to a pharmaceutical composition comprising an effective amount of a MayDay polypeptide as contemplated herein, such as an effective amount of a MayDay polypeptide described herein or a pharmaceutically acceptable salt or solvent thereof, and a pharmaceutically acceptable carrier.

While the following discussion focuses on pharmaceutical compositions, those skilled in the art will appreciate that this discussion applies also to the preparation of medicaments for treatment of the diseases or conditions contemplated herein.

The pharmaceutical compositions may comprise an effective amount of two or more agents, such as two or more MayDay polypeptides described herein, in combination.

Compositions suitable for the administration of bioactive agents, such as a bioactive protein, including the therapeutic administration of a bioactive polypeptides to a subject in need thereof, are known in the art.

For example, the bioactive MayDay polypeptide as described herein, or a fragment, a variant, a peptide analogue, or a derivative thereof, is in certain examples formulated with a pharmaceutically acceptable carrier, excipient or combined with other agents to improve bioavailability, or half-life, or potency of the bioactive, and the composition is administered to the subject.

The term “pharmaceutically acceptable carrier” refers to a carrier (adjuvant or vehicle) that may be administered to a subject together with the bioactive agent, such as the MayDay peptide described herein, or a pharmaceutically acceptable salt or solvate thereof.

Once such carrier is saline (0.9% sodium chloride), though other carriers are suitable.

Pharmaceutically acceptable carriers that may be used in the compositions include, but are not limited to, ion exchangers, alumina, aluminum stearate, lecithin, self-emulsifying drug delivery systems (SEDDS) such as d-a-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Cyclodextrins such as α-, β-, and γ-cyclodextrin, or chemically modified derivatives such as hydroxyalkylcyclodextrins, including 2- and 3-hydroxypropyl-3-cyclodextrins, or other solubilized derivatives may also be advantageously used to enhance delivery. Oil solutions or suspensions may also contain a long-chain alcohol diluent or dispersant, or carboxymethyl cellulose or similar dispersing agents, which are commonly used in the formulation of pharmaceutically acceptable dosage forms such as emulsions and or suspensions.

The compositions are formulated to allow for administration to a subject by any chosen route, including but not limited to oral or parenteral (including topical, subcutaneous, intramuscular, and intravenous) administration.

For example, the compositions may be formulated with an appropriate pharmaceutically acceptable carrier (including excipients, diluents, auxiliaries, and combinations thereof) selected with regard to the intended route of administration and standard pharmaceutical practice.

Accordingly, in various examples, the carrier is selected from the group comprising a diluent or an excipient.

In various examples, the compositions may be administered orally as a powder, liquid, tablet or capsule, or topically as an ointment, cream or lotion. Suitable formulations may contain additional agents as required, including emulsifying, antioxidant, flavouring or colouring agents, and may be adapted for immediate-, delayed-, modified-, sustained-, pulsed- or controlled-release.

In certain examples involving the administration of a polypeptide as described herein, administration will typically involve injection or deposition directly into a site of interest, for example, into soft tissue at the site of an injury. Parenteral administration is accordingly contemplated.

The compositions may be formulated to optimize bioavailability or activity, or to maintain plasma, blood, or tissue concentrations within the therapeutic range, including for extended periods. Controlled delivery preparations may also be used to optimize the bioactive agent concentration at the site of action, for example.

The compositions may be formulated for periodic administration, for example to provide continued exposure.

The compositions may be administered via the parenteral route. Examples of parenteral dosage forms include aqueous solutions, isotonic saline or 5% glucose of the active agent, or other well-known pharmaceutically acceptable excipients. Cyclodextrins, for example, or other solubilising agents well-known to those familiar with the art, can be utilized as pharmaceutical excipients for delivery of the therapeutic agent.

Examples of dosage forms suitable for oral administration include, but are not limited to tablets, capsules, lozenges, or like forms, or any liquid forms such as syrups, aqueous solutions, emulsions and the like, capable of providing a therapeutically effective amount of the composition. Capsules can contain any standard pharmaceutically acceptable materials such as gelatin or cellulose. Tablets can be formulated in accordance with conventional procedures by compressing mixtures of the active ingredients with a solid carrier and a lubricant. Examples of solid carriers include starch and sugar bentonite.

Active ingredients can also be administered in a form of a hard shell tablet or a capsule containing a binder, e.g., lactose or mannitol, a conventional filler, and a tabletting agent. Dosage forms for oral administration can be formulated with an enteric coating to prevent dissolution or disintegration of the dosage form in the stomach to provide for delayed release of the agent and/or to allow release of the agent after the stomach (such as in the upper tract of the intestine).

Examples of dosage forms suitable for transdermal administration include, but are not limited, to transdermal patches, transdermal bandages, and the like.

Examples of dosage forms suitable for topical administration of the compositions include any lotion, stick, spray, ointment, paste, cream, gel, etc., whether applied directly to the skin or via an intermediary such as a pad, patch or the like.

Examples of dosage forms suitable for suppository administration of the compositions include any solid dosage form inserted into a bodily orifice particularly those inserted rectally, vaginally and urethrally.

Examples of dosage of forms suitable for injection of the compositions include delivery via bolus such as single or multiple administrations by intravenous injection, subcutaneous, subdermal, and intramuscular administration, or oral administration.

Examples of dosage forms suitable for depot administration of the compositions include pellets of the peptide or solid forms wherein the peptide is entrapped in a matrix of biodegradable polymers, microemulsions, liposomes or are microencapsulated.

Examples of infusion devices for the compositions include infusion pumps for providing a desired number of doses or steady state administration, and include implantable drug pumps.

Examples of implantable infusion devices for compositions include any solid form in which the bioactive agent is encapsulated within or dispersed throughout a biodegradable polymer or synthetic polymer such as silicone, silicone rubber, silastic or similar polymer.

Examples of dosage forms suitable for transmucosal delivery of the compositions include depositories solutions for enemas, pessaries, tampons, creams, gels, pastes, foams, nebulised solutions, powders and similar formulations containing in addition to the active ingredients such carriers as are known in the art to be appropriate. Such dosage forms include forms suitable for inhalation or insufflation of the compositions, including compositions comprising solutions and/or suspensions in pharmaceutically acceptable, aqueous, or organic solvents, or mixture thereof and/or powders. Transmucosal administration of the compositions may utilize any mucosal membrane but commonly utilizes the nasal, buccal, vaginal and rectal tissues. Formulations suitable for nasal administration of the compositions may be administered in a liquid form, for example, nasal spray, nasal drops, or by aerosol administration by nebulizer, including aqueous or oily solutions of the polymer particles. Formulations may be prepared as aqueous solutions for example in saline, solutions employing benzyl alcohol or other suitable preservatives, absorption promoters to enhance bioavailability, fluorocarbons, and/or other solubilising or dispersing agents known in the art.

Examples of dosage forms suitable for buccal or sublingual administration of the compositions include lozenges, tablets and the like. Examples of dosage forms suitable for opthalmic administration of the compositions include inserts and/or compositions comprising solutions and/or suspensions in pharmaceutically acceptable, aqueous, or organic solvents.

Examples of formulations of compositions may be found in, for example, Sweetman, S. C. (Ed.). Martindale. The Complete Drug Reference, 33rd Edition, Pharmaceutical Press, Chicago, 2002, 2483 pp.; Aulton, M. E. (Ed.) Pharmaceutics. The Science of Dosage Form Design. Churchill Livingstone, Edinburgh, 2000, 734 pp.; and, Ansel, H. C, Allen, L. V. and Popovich, N. G. Pharmaceutical Dosage Forms and Drug Delivery Systems, 7th Ed., Lippincott 1999, 676 pp. Excipients employed in the manufacture of drug delivery systems are described in various publications known to those skilled in the art including, for example, Kibbe, E. H. Handbook of Pharmaceutical Excipients, 3rd Ed., American Pharmaceutical Association, Washington, 2000, 665 pp. The USP also provides examples of modified-release oral dosage forms, including those formulated as tablets or capsules. See, for example, The United States Pharmacopeia 23/National Formulary 18, The United States Pharmacopeial Convention, Inc., Rockville MD, 1995 (hereinafter “the USP”), which also describes specific tests to determine the drug release capabilities of extended-release and delayed-release tablets and capsules. The USP test for drug release for extended-release and delayed-release articles is based on drug dissolution from the dosage unit against elapsed test time. Descriptions of various test apparatus and procedures may be found in the USP. Further guidance concerning the analysis of extended release dosage forms has been provided by the F.D.A. (See Guidance for Industry. Extended release oral dosage forms: development, evaluation, and application of in vitro/in vivo correlations. Rockville, MD: Center for Drug Evaluation and Research, Food and Drug Administration, 1997).

Where two or more agents are administered or used, the two or more agents may be administered or used simultaneously, sequentially, or separately.

The invention is further described with reference to the following examples. It will be appreciated that the invention as claimed is not intended to be limited in any way by these examples.

### EXAMPLES

### Example 1: Therapeutic Efficacy of MayDay Polypeptide

This example presents an assessment of the therapeutic efficacy of administration of a MayDay polypeptide as described herein in an animal model of endogenous stem cell recruitment.

### Materials and Methods

### Ethics Statement

Animal manipulations were carried out in accordance with the recommendations in the Guide for the Care and Use of Laboratory Animals of the Society of Laboratory Animals (GV SOLAS) in an AAALAC accredited animal facility. All animal experiments were approved by the Committee on the Ethics of Animal Experiments of the regional council (Regierungspräsidium Freiburg, Abt. Landwirtschaft, Ländlicher Raum, Veterinär-und Lebensmittelwesen—Ref. 35, permit #: G-21/100). All animal experiments were performed in accordance with the guidelines of decreasing the amount of suffering, pain, and discomfort of the experimental animals. Animals were acclimated for at least 4 days prior to the study. Animals were housed in a temperature-controlled room with a 12-hour light/dark cycle, with ad libitum access to water and irradiated laboratory chow throughout the study. Animals were individually identified by ear tags. Animals were monitored for any form of discomfort as well as for clinical symptoms of distress or ambulatory discomfort.

### Recombinant MayDay Polypeptide Expression and Purification

A representative recombinant MayDay polypeptide, MayDay₃₁₋₁₇₀ (MD₃₁₋₁₇₀) comprising amino acids 31-170 of ovine decorin, was expressed using a pSUMO vector in BL21 E. coli. After expansion, expression and lysis, supernatant was loaded onto a Q Sepharose™ for purification. Purity (>85%) was confirmed by SDS-PAGE.

### Endogenous Recruitment of Stem Cells by Intramuscular Injection of MD₃₁₋₁₇₀

A total of 105 immunocompetent, tumour free Balb/c mice were allocated to five treatment groups of 21 animals for sampling at three times points (n=7 per group per timepoint). Test articles for intramuscular injection of MD₃₁₋₁₇₀ and human recombinant SDF-1 (Sigma) were prepared in 0.9% sterile saline (Braun, Melsungen, Germany). Five treatment groups were used; MD₃₁₋₁₇₀ 1 (1 μg/animal or 0.06 μg/kg), MD₃₁₋₁₇₀ 10 (10 μg/animal or 0.6 mg/kg), MD₃₁₋₁₇₀ 100 (100 μg/animal or 6 mg/kg), SDF1 control (10 μg/animal or 0.5 mg/kg) and 0.9% sterile saline control. Intramuscular injection sites (right side hindlimb) were prepared with chlorhexidine wipes before test articles were administered via a 100 μL intramuscular injection to the right hindlimb muscle (‘treated’ side). Upon treatment the injection site was marked with a surgery pen to relocate it for sampling. For each group animals were anesthetized at one of three sampling endpoints (24, 48 and 72 hours after injection) using isoflurane and placed in a ventral recumbency with anesthetic gas administered via nose cone.

### Isolation of Muscle Biopsies

Treated hindlimb muscle tissue was excised using a 10 mm punch biopsy. Each biopsy was bisected through the centre and one half was fixed in 4% formalin for 24 h and then stored in 70% ethanol for immunohistochemistry (IHC) staining. The second half of the biopsied tissue was processed directly for flow cytometry (FACS) analysis.

### Flow Cytometry of Muscle Tissue

For flow cytometry analysis, single cell suspensions of the muscle tissues were prepared using a tumour dissociation kit according to manufacturer's instructions (Miltenyi Biotec, mouse TDK #130-096-730). Briefly, muscles were cut into small pieces (2-4 mm), then transferred into a dissociation tube (GentleMACS), incubated with “enzyme mixture” from the kit and processed in the dissociator. Samples were then strained through a 100 μm mesh to obtain single cells. Cells were pelleted by centrifugation at 400×g for 5 min and the supernatant was removed. The cells were washed (2×) by resuspension in a staining buffer of 2% fetal bovine serum (FBS) in phosphate buffered saline (PBS) and centrifuged at 300×g for 5 min. Cells were resuspended in staining buffer, counted and processed for flow cytometry analysis.

Flow cytometry analysis of muscle biopsies was carried out using the Mouse Mesenchymal Stromal Cell 4-Color Flow Cytometry Kit (R&DSystems (Biotechne) Catalog #FMC003) to identify cells positive for the stem cell markers CD105, CD29, and SCA1 and negative for CD45 using the following antibodies: Rat IgG2A Anti-Mouse Endoglin/CD105-Fluorescein (Clone 209701), Rat IgG2A Anti-Mouse Integrin beta 1/CD29-PE (Clone 265917), Rat IgG2A Anti-Mouse SCA1-APC (Clone 177228). Briefly, muscle cells were transferred to a 96-well plate (3×10⁵ cells/well) and were pelleted by centrifugation of the plates at 400×g for 5 min and the supernatant was removed. Fc-block antibody (10 μL/well of a 1:100 dilution in staining buffer) was added to each well and plates were incubated for 5 min at room temperature. Specific antibodies or isotype controls were then added as recommended by the manufacturer, and the plates were incubated for 30 min at 4° C. protected from light. Cells were washed by the addition of 200 μL staining buffer followed by centrifugation of the plates at 400×g for 5 min and the supernatant was removed. Samples were resuspended in staining buffer for analysis with the Attune NXT Acoustic Focusing Cytometer (violet (405 nm)/blue (488 nm)/yellow (561 nm)/red (638 nm) laser configuration).

Flow cytometry data were analyzed with FlowJo Data Analysis Software (version 10.8.1) using fluorescence minus one (FMO) to establish accurate gating and relevant isotype controls to detect and exclude unspecific background signals. Doublet exclusion was performed according to forward scatter height versus forward scatter area to include only single cells, followed by a live/dead discrimination. The software automatically determined the frequency of subpopulations in percent relative to the parent population.

### SCA1+ Immunohistochemistry of Muscle Tissue

Formalin fixed samples of the muscle biopsies from the treated (right) hindlimb of each animal were dehydrated and embedded in paraffin according to standard histological procedures. Each half punch biopsy was oriented on its central cutting plane (the longest edge) to achieve uniform orientation of samples. Consecutive 5 μm thick sections spaced 0.1 mm apart (n=3 sections per tissue sample) were prepared from each FFPE block for SCA1+ staining and haematoxylin counter staining. Slides were processed using an automated staining system (Autostainer Link 24, Agilent, CA, USA) using recombinant monoclonal rabbit anti-SCA1 (Ly6A/E), Abcam ([EPR22502-68], ab255604) and an anti rabbit detection system EnVision rabbit (Dako Cat #K4003, Lot: 11337301). Whole sections were scanned using (NanoZoomer S60 Digital slide scanner, Hamamatsu, Japan) and high power field of view (HPFV) images at ×100 magnification were exported from each scanned section.

A total of nine evenly spaced images were captured from each of the three sections taken from each tissue sample (n=27 images per animal, 7 animals per group). In total, 189 images per group, per timepoint were analysed by an automated ImageJ (NIH) macro to quantify the average SCA1+ cell count per HPFV.

### Statistical Analysis

Figures were generated and statistical analysis was performed with GraphPad Prism software version 9.2.0 (San Diego, USA). All data were presented as mean±standard deviation (SD). Statistical comparisons between pairs of groups were measured by ANOVA with P values for significance set to 0.05 for all tests.

### Results

### Flow Cytometry Analysis of Muscle Tissue

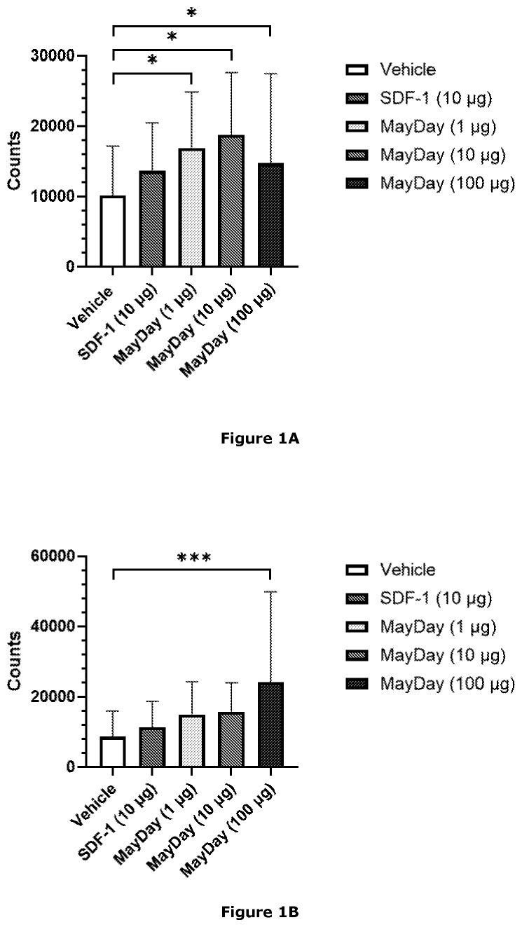

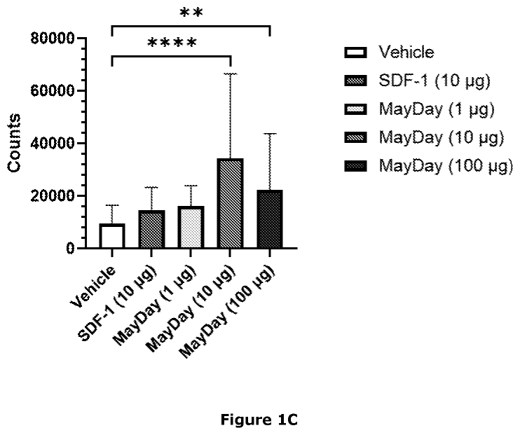

Hindlimb muscle tissue was analysed for the expression of three stem cell markers (SCA1, CD29 and CD105) 72 hours after intramuscular injection of controls and test articles. Results for each marker were expressed as “counts” to compare stem cell marker expression between different treatment groups and controls. FIG. 1 displays the average stem cell “counts” using the markers CD29 (FIG. 1A); CD105 (FIG. 1B) and SCA1 (FIG. 1C) at the 72 h endpoint. Error bars indicate the standard deviation of each group (n=7).

These data demonstrate that the administration of MD₃₁₋₁₇₀ is effective in increasing the number of SCA1-positive stem cells at each of the time points assessed.

These data also show that 72 hours after IM injection of MD₃₁₋₁₇₀ an increase in stem cells expressing one of the stem cell markers SCA1, CD29 and CD105 was observed within the tissue to which MD₃₁₋₁₇₀ had been administered.

Administration of MD₃₁₋₁₇₀ elicited a significant increase in the number of cells expressing these stem cell markers. Thus, the administration of MayDay polypeptide is effective in recruiting cells expressing stem cell markers to specific tissues in this murine model of stem cell recruitment.

### IHC Analysis of Muscle Tissue

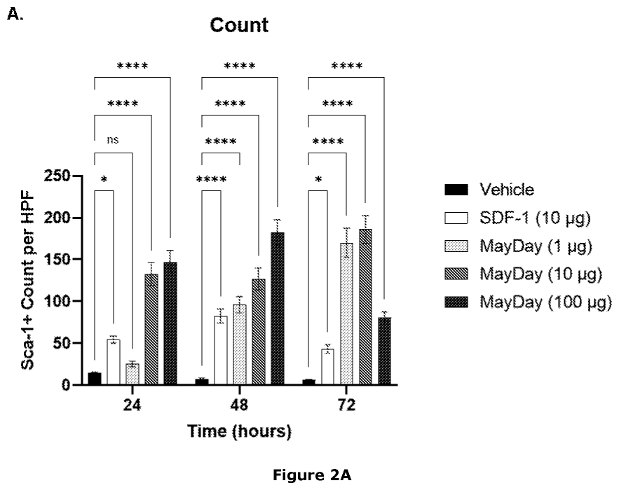

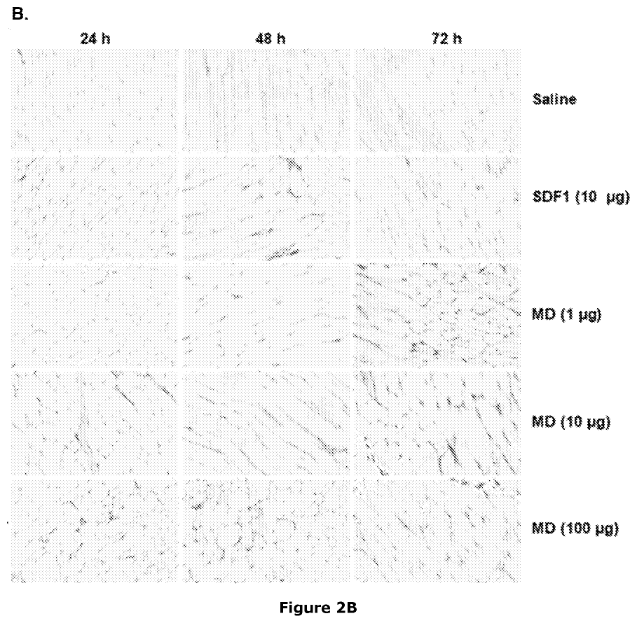

IHC was used to enumerate the number of SCA1+ cells within the muscle tissue after injection with test articles and controls. Images were taken to show SCA1+ cells in brown with purple counterstaining of muscle tissue in the background. SCA1+ cells were quantified at 3 levels of each tissue sample to give an average cell count per HPFV. The average SCA1+ cell count per HPFV (with error bars indicating SD between groups) at each of the 24 h, 48 h, and 72 h endpoints is shown in FIG. 2A. FIG. 2B shows an exemplary image from each group and timepoint after SCA1 staining.

### Discussion

These results indicate that representative MayDay polypeptides can effectively increase the number of stem cells in tissue in an animal model of stem cell recruitment. This in turn supports the use of MayDay polypeptides in methods of treating or preventing diseases or conditions associated with tissue ischemia.

### Example 2: Therapeutic Efficacy of MayDay Polypeptide in Myocardial Infarction

This example presents an assessment of the therapeutic efficacy of the administration of a MayDay polypeptide as described herein in an animal model of myocardial infarction (MI).

### Materials and Methods

A total of 21 SD rats, aged 8 weeks old were used in this study. Animals were housed in a temperature-controlled room with a 12-hour light/dark cycle, with ad libitum access to water and irradiated laboratory chow throughout the study. Animals were individually identified by ear tags.

Animals were weighed prior to the start of the study and divided into 2 groups: group 1 control group (n=7); group 2, treatment (n=7). Animals were monitored for any form of discomfort. At day 0 (T=0), myocardial infarction (MI) was induced by surgical ligation of the left anterior descending (LAD) coronary artery. At the time of surgical ligation, animals were treated by intramyocardial injection across four sites (25 μL per site) adjacent to the ischemic zone with saline (vehicle control) (group 1, n=7), test article (100 μg MD₃₁₋₁₇₀ dissolved in 100 μL saline) (group 2, n=7) according to Table 2.

TABLE 2
Experimental design

| | | | MI | | |
|---|---|---|---|---|---|
| Group | N | Description | Induction | Treatment | Sampling |
| 1 | 7 | Healthy animal group | No ligation | No treatment | Echocardiography |
| 1 | 7 | Vehicle control group | Surgical | 4 injections (25 μL) | (ESV, EDV) |
| | | (saline) | ligation | of saline | |
| 2 | 7 | Treatment group (100 μg | Surgical | 4 injections (25 μL) | |
| | | MD₃₁₋₁₇₀ dissolved in 100 | ligation | MD₃₁₋₁₇₀ in saline | |
| | | μL saline) | | | |

Animals were anesthetized with isofluorane for echocardiography measurements at 4 weeks and 8 weeks after induction of MI. End-systolic volume (ESV) and end-diastolic volume (EDV) were measured by echocardiograph using an 18 MHz MicroScan transducer and cardiovascular ultrasound system (Vevo 2100 Imaging System, VisuialSonics). Animals were anesthetized and cardiac tissue was collected for gross pathological examination. The tissue was fixed in paraformaldehyde (4%). Fixed, cardiac tissues were embedded in paraffin and then cut into 4 μm sections. Sections were deparaffinized and stained with hematoxylin and eosin (H&E). The severity of tissue injury was evaluated in terms of collagen deposition, fibrosis, mononuclear infiltration, myocardiocytes and scarring.

### Results

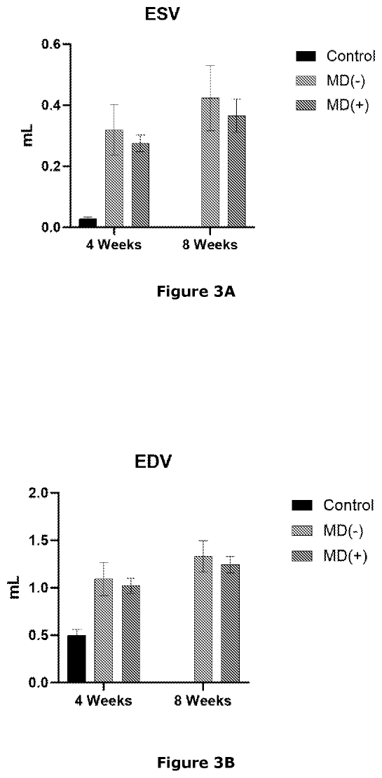

To explore the therapeutic potential of MayDay (MD) in an MI model in vivo, an established rat surgical ligation model was performed followed by treatment in intracardiac injections of saline MD₃₁₋₁₇₀ (−) and MD₃₁₋₁₇₀ treatment group MD₃₁₋₁₇₀ (+). ESV and EDV were measured at 4 weeks and 8 weeks after MI induction in a healthy animal (control), vehicle control MD₃₁₋₁₇₀ (−) and treated group MD₃₁₋₁₇₀ (+) as shown in FIG. 3. ESV and EDV in non-treated animals were lower than in animals with induced ischemia, and there was an improvement in the MD₃₁₋₁₇₀ (+) group.

### Discussion

These results show that the administration of a representative MayDay polypeptide can effectively improve post-MI echocardiography in an animal model of myocardial infarction. This in turn supports the use of MayDay polypeptides in methods of treating or preventing diseases or conditions associated with tissue ischemia, such as myocardial infarction.

### PUBLICATIONS

Berry, M. F., Engler, A. J., Woo, Y. J., Pirolli, T. J., Bish, L. T., Jayasankar, V., & Sweeney, H. L. (2006). Mesenchymal stem cell injection after myocardial infarction improves myocardial compliance. American Journal of Physiology-Heart and Circulatory Physiology, 290 (6), H2196-H2203.
Bolli, R., Solankhi, M., Tang, X.-L., Kahlon, A. (2022). Cell therapy in patients with heart failure: a comprehensive review and emerging concepts. Cardiovascular Research, Volume 118, Issue 4, March 2022, Pages 951-976.
di Summa, P. G., Kingham, P. J., Raffoul, W., Wiberg, M., Terenghi, G., & Kalbermatten, D. F. (2010). Adipose-derived stem cells enhance peripheral nerve regeneration. Journal of Plastic, Reconstructive & Aesthetic Surgery, 63 (9), 1544-1552.
Dempsey, S. G., C. H. Miller, J. Schueler, R. W. F. Veale, D. J. Day and B. C. H. May (2020). “A novel chemotactic factor derived from the extracellular matrix protein decorin recruits mesenchymal stromal cells in vitro and in vivo.” PLoS One 15 (7): e0235784.
Duscher, D., Barrera, J., Wong, V. W., Maan, Z. N., Whittam, A. J., Januszyk, M., & Gurtner, G. C. (2016). Stem cells in wound healing: the future of regenerative medicine? A mini-review. Gerontology, 62 (2), 216-225.].
Falanga, V., Iwamoto, S., Chartier, M., Yufit, T., Butmarc, J., Kouttab, N., & Carson, P. (2007). Autologous bone marrow-derived cultured mesenchymal stem cells delivered in a fibrin spray accelerate healing in murine and human cutaneous wounds. Tissue engineering, 13 (6), 1299-1312.
Kanji, S., & Das, H. (2017). Advances of stem cell therapeutics in cutaneous wound healing and regeneration. Mediators of inflammation, 2017.
Li, L., & Xie, T. (2005). Stem cell niche: structure and function. Annu. Rev. Cell Dev. Biol., 21, 605-631.

As used in this specification, the words “comprise”, “comprises”, “comprising”, and similar words, are not to be interpreted in an exclusive or exhaustive sense. In other words, they are intended to mean “including, but not limited to”. When interpreting each statement in this specification that includes the term “comprise”, “comprises”, or “comprising”, features other than that or those prefaced by the term may also be present.

The entire disclosures of all applications, patents and publications cited above and below, if any, are herein incorporated by reference.

Where in the foregoing description reference has been made to integers or components having known equivalents thereof, those integers are herein incorporated as if individually set forth.

It should be noted that various changes and modifications to the presently preferred examples described herein will be apparent to those skilled in the art. Such changes and modifications may be made without departing from the spirit and scope of the invention and without diminishing its attendant advantages. It is therefore intended that such changes and modifications be included within the present invention.

The invention may also be said broadly to consist in the parts, elements and features referred to or indicated in the specification of the application, individually or collectively, in any or all combinations of two or more of said parts, elements or features.

Aspects of the invention have been described by way of example only, and it should be appreciated that variations, modifications and additions may be made without departing from the scope of the invention, for example when claims (indicative or otherwise) are present the invention as defined in those claims. Furthermore, where known equivalents exist to specific features, such equivalents are incorporated as if specifically referred to in this specification.

SEQUENCE LISTING

Sequence Listing Information:
DTD Version: V1_3
File Name: MES1025_PC.xml
Software Name: WIPO Sequence
Software Version: 2.3.0
Production Date: 2024-01-16
General Information:
Current application/IP Office: WO
Current application/Applicant file reference: MES1025/PC
Earliest priority application/IP Office: AU
Earliest priority application/Application number: 2023900186
Earliest priority application/Filing date: 2023-01-27
Applicant name: Aroa Biosurgery Limited
Applicant name/Language: en
Invention title: METHODS AND AGENTS FOR TREATING TISSUE ISCHEMIA AND ASSOCIATED
DISEASES AND CONDITIONS (en)
Sequence Total Quantity: 13
Sequences:
Sequence Number (ID): 1
Length: 360
Molecule Type: AA
Features Location/Qualifiers:
-source, 1..360
>mol_type, protein
>organism, Ovis aries
Residues:

| | |
|---|---|
| MKATIIFFLV AQVSWAGPFQ QKGLFDFMLE DEASGIGPEE RFHEVPELEP MGPVCPFRCQ | 60 |
| CHLRVVQCSD LGLEKVPKDL PPDTALLDLQ NNKITEIKDG DFKNLKNLHT LILINNKISK | 120 |
| ISPGAFAPLV KLERLYLSKN QLKELPEKMP KTLQELRVHE NEITKVRKSV FNGLNQMIVV | 180 |
| ELGTNPLKSS GIENGAFQGM KKLSYIRIAD TNITTIPQGL PPSLTELHLD GNKITKVDAA | 240 |
| SLKGLNNLAK LGLSFNSISA VDNGSLANTP HLRELHLNNN KLVKVPGGLA DHKYIQVVYL | 300 |
| HNNNISAIGS NDFCPPGYNT KKASYSGVSL FSNPVQYWEI QPSTFRCVYV RAAVQLGNYK | 360 |

Sequence Number (ID): 2
Length: 140
Molecule Type: AA
Features Location/Qualifiers:
-source, 1..140
>mol_type, protein
>organism, Ovis aries
Residues:

| | |
|---|---|
| DEASGIGPEE RFHEVPELEP MGPVCPFRCQ CHLRVVQCSD LGLEKVPKDL PPDTALLDLQ | 60 |
| NNKITEIKDG DFKNLKNLHT LILINNKISK ISPGAFAPLV KLERLYLSKN QLKELPEKMP | 120 |
| KTLQELRVHE NEITKVRKSV | 140 |

Sequence Number (ID): 3
Length: 147
Molecule Type: AA
Features Location/Qualifiers:
-source, 1..147
>mol_type, protein
>organism, Ovis aries
Residues:

| | |
|---|---|
| DEASGIGPEE RFHEVPELEP MGPVCPFRCQ CHLRVVQCSD LGLEKVPKDL PPDTALLDLQ | 60 |
| NNKITEIKDG DFKNLKNLHT LILINNKISK ISPGAFAPLV KLERLYLSKN QLKELPEKMP | 120 |
| KTLQELRVHE NEITKVRKSV FNGLNQM | 147 |

Sequence Number (ID): 4
Length: 158
Molecule Type: AA
Features Location/Qualifiers:
-source, 1..158
>mol_type, protein
>organism, Ovis aries
Residues:

| | |
|---|---|
| DEASGIGPEE RFHEVPELEP MGPVCPFRCQ CHLRVVQCSD ISPGAFAPLV NNKITEIKDG | 60 |
| NNKITEIKDG DFKNLKNLHT LILINNKISK ISPGAFAPLV KLERLYLSKN QLKELPEKMP | 120 |
| KTLQELRVHE NEITKVRKSV FNGLNQMIVV ELGTNPLK | 158 |

Sequence Number (ID): 5
Length: 359
Molecule Type: AA
Features Location/Qualifiers:
-source, 1..359
>mol_type, protein
>organism, Homo sapiens
Residues:

| | |
|---|---|
| MKATIILLLL AQVSWAGPFQ QRGLFDFMLE DEASGIGPEV PDDRDFEPSL GPVCPFRCQC | 60 |
| HLRVVQCSDL GLDKVPKDLP PDTTLLDLQN NKITEIKDGD FKNLKNLHAL ILVNNKISKV | 120 |
| SPGAFTPLVK LERLYLSKNQ LKELPEKMPK TLQELRAHEN EITKVRKVTF NGLNQMIVIE | 180 |
| LGTNPLKSSG IENGAFQGMK KLSYIRIADT NITSIPQGLP PSLTELHLDG NKISRVDAAS | 240 |
| LKGLNNLAKL GLSFNSISAV DNGSLANTPH LRELHLDNNK LTRVPGGLAE HKYIQVVYLH | 300 |
| NNNISVVGSS DFCPPGHNTK KASYSGVSLF SNPVQYWEIQ PSTFRCVYVR SAIQLGNYK | 359 |

Sequence Number (ID): 6
Length: 139
Molecule Type: AA
Features Location/Qualifiers:
-source, 1..139
>mol_type, protein
>organism, Homo sapiens
Residues:

| | |
|---|---|
| DEASGIGPEV PDDRDFEPSL GPVCPFRCQC HLRVVQCSDL GLDKVPKDLP PDTTLLDLQN | 60 |
| NKITEIKDGD FKNLKNLHAL ILVNNKISKV SPGAFTPLVK LERLYLSKNQ LKELPEKMPK | 120 |
| TLQELRAHEN EITKVRKVT | 139 |

Sequence Number (ID): 7
Length: 157
Molecule Type: AA
Features Location/Qualifiers:
-source, 1..157
>mol_type, protein
>organism, Homo sapiens
Residues:

| | |
|---|---|
| DEASGIGPEV PDDRDFEPSL GPVCPFRCQC HLRVVQCSDL GLDKVPKDLP PDTTLLDLQN | 60 |
| NKITEIKDGD FKNLKNLHAL ILVNNKISKV SPGAFTPLVK LERLYLSKNQ LKELPEKMPK | 120 |
| TLQELRAHEN EITKVRKVTF NGLNQMIVIE LGTNPLK | 157 |

Sequence Number (ID): 8
Length: 354
Molecule Type: AA
Features Location/Qualifiers:
-source, 1..354
>mol_type, protein
>organism, Mus musculus
Residues:

| | |
|---|---|
| MKATLIFFLL AQVSWAGPFE QRGLFDFMLE DEASGIIPYD PDNPLISMCP YRCQCHLRVV | 60 |
| QCSDLGLDKV PWDFPPDTTL LDLQNNKITE IKEGAFKNLK DLHTLILVNN KISKISPEAF | 120 |
| KPLVKLERLY LSKNQLKELP EKMPRTLQEL RVHENEITKL RKSDFNGLNN VLVIELGGNP | 180 |
| LKNSGIENGA FQGLKSLSYI RISDTNITAI PQGLPTSLTE VHLDGNKITK VDAPSLKGLI | 240 |
| NLSKLGLSFN SITVMENGSL ANVPHLRELH LDNNKLLRVP AGLAQHKYIQ VVYLHNNNIS | 300 |
| AVGQNDFCRA GHPSRKASYS AVSLYGNPVR YWEIFPNTFR CVYVRSAIQL GNYK | 354 |

Sequence Number (ID): 9
Length: 134
Molecule Type: AA
Features Location/Qualifiers:
-source, 1..134
>mol_type, protein
>organism, Mus musculus
Residues:

| | |
|---|---|
| DEASGIIPYD PDNPLISMCP YRCQCHLRVV QCSDLGLDKV PWDFPPDTTL LDLQNNKITE | 60 |
| IKEGAFKNLK DLHTLILVNN KISKISPEAF KPLVKLERLY LSKNQLKELP EKMPRTLQEL | 120 |
| RVHENEITKL RKSD | 134 |

Sequence Number (ID): 10
Length: 152
Molecule Type: AA
Features Location/Qualifiers:
-source, 1..152
>mol_type, protein
>organism, Mus musculus
Residues:

| | |
|---|---|
| DEASGIIPYD PDNPLISMCP YRCQCHLRVV QCSDLGLDKV PWDFPPDTTL LDLQNNKITE | 60 |
| IKEGAFKNLK DLHTLILVNN KISKISPEAF KPLVKLERLY LSKNQLKELP EKMPRTLQEL | 120 |
| RVHENEITKL RKSDFNGLNN VLVIELGGNP LK | 152 |

Sequence Number (ID): 11
Length: 360
Molecule Type: AA
Features Location/Qualifiers:
-source, 1..360
>mol_type, protein
>organism, Bos taurus
Residues:

| | |
|---|---|
| MKATIIFLLV AQVSWAGPFQ QKGLFDFMLE DEASGIGPEE HFPEVPEIEP MGPVCPFRCQ | 60 |
| CHLRVVQCSD LGLEKVPKDL PPDTALLDLQ NNKITEIKDG DFKNLKNLHT LILINNKISK | 120 |
| ISPGAFAPLV KLERLYLSKN QLKELPEKMP KTLQELRVHE NEITKVRKSV FNGLNQMIVV | 180 |
| ELGTNPLKSS GIENGAFQGM KKLSYIRIAD TNITTIPQGL PPSLTELHLD GNKITKVDAA | 240 |
| SLKGLNNLAK LGLSFNSISA VDNGSLANTP HLRELHLNNN KLVKVPGGLA DHKYIQVVYL | 300 |
| HNNNISAIGS NDFCPPGYNT KKASYSGVSL FSNPVQYWEI QPSTFRCVYV RAAVQLGNYK | 360 |

Sequence Number (ID): 12
Length: 140
Molecule Type: AA
Features Location/Qualifiers:
-source, 1..140
>mol_type, protein
>organism, Bos taurus
Residues:

| | |
|---|---|
| DEASGIGPEE HFPEVPEIEP MGPVCPFRCQ CHLRVVQCSD LGLEKVPKDL PPDTALLDLQ | 60 |
| NNKITEIKDG DFKNLKNLHT LILINNKISK ISPGAFAPLV KLERLYLSKN QLKELPEKMP | 120 |
| KTLQELRVHE NEITKVRKSV | 140 |

Sequence Number (ID): 13
Length: 158
Molecule Type: AA
Features Location/Qualifiers:
-source, 1..158
>mol_type, protein
>organism, Bos taurus
Residues:

| | |
|---|---|
| DEASGIGPEE HFPEVPEIEP MGPVCPFRCQ CHLRVVQCSD LGLEKVPKDL PPDTALLDLQ | 60 |
| NNKITEIKDG DFKNLKNLHT LILINNKISK ISPGAFAPLV KLERLYLSKN QLKELPEKMP | 120 |
| KTLQELRVHE NEITKVRKSV FNGLNQMIVV ELGTNPLK | 158 |

END

1. A method of treating or preventing tissue ischemia in a subject in need thereof, the method comprising administering to the subject a therapeutically effective amount of a MayDay polypeptide.

2. The method of claim 1, wherein the subject has or has an increased risk of having or is predicted to have an increased risk of having a disease or condition associated with tissue ischemia.

3. A method of treating or preventing a disease or condition associated with tissue ischemia in a subject in need thereof, the method comprising administering to the subject a therapeutically effective amount of a MayDay polypeptide.

4. The method according to claim 2 or 3, wherein the disease or condition associated with tissue ischemia is a disease or condition selected from the group consisting of: ischemic-reperfusion injury in an organ for transplantation, spinal cord injury, myocardial infarction, heart failure, acute kidney injury, renal failure, ischemic stroke, hemorrhagic stroke, acute tubular necrosis, compartment syndrome, coronary artery disease, intestinal ischemia, peripheral artery disease, transient ischemic attack, ischemia due to atherosclerosis, chronic kidney disease, pancreatic ischemia, critical limb ischemia, chronic wounds, cirrhosis, liver failure, liver ischemia, vascular dementia, cutaneous ischemia, ischemia due to thrombosis, ischemia due to embolism, ischemia due to trauma, ischemia due to aneurysm, and traumatic brain injury.

5. The method of claim 4, wherein the disease or condition is selected from the group consisting of: myocardial infarction, heart failure, and stroke.

6. The method of claim 4, wherein the disease or condition is selected from the group consisting of: acute kidney injury, acute kidney disease, renal failure, and chronic wounds.

7. A method of treating or preventing myocardial infarction, heart failure, and/or cardiomyopathy in a subject in need thereof, the method comprising administering to the subject a therapeutically effective amount of a MayDay polypeptide.

8. The method according to any one of claims 1 to 7, wherein the MayDay polypeptide is an isolated, purified, recombinant or synthetic polypeptide selected from the group comprising:
a) a polypeptide comprising, consisting essentially of, or consisting of an amino acid sequence corresponding to residues 1 to 188 of mammalian Decorin;
b) a polypeptide comprising, consisting essentially of, or consisting of an amino acid sequence corresponding to residues 31 to 188 of mammalian Decorin;
c) an N-terminal fragment of mammalian Decorin comprising, consisting essentially of, or consisting of at least 10 contiguous amino acids corresponding to any amino acid sequence within residues 1 to 188 of mammalian Decorin;
d) an N-terminal fragment of mammalian Decorin comprising, consisting essentially of, or consisting of at least 10 contiguous amino acids corresponding to any amino acid sequence within residues 31 to 188 of mammalian Decorin;
e) a polypeptide comprising, consisting essentially of, or consisting of an amino acid sequence corresponding to residues 1 to 177 of mammalian Decorin;
f) a polypeptide comprising, consisting essentially of, or consisting of an amino acid sequence corresponding to residues 31 to 177 of mammalian Decorin;
g) an N-terminal fragment of mammalian Decorin comprising, consisting essentially of, or consisting of at least 10 contiguous amino acids corresponding to any amino acid sequence within residues 1 to 177 of mammalian Decorin;
h) an N-terminal fragment of mammalian Decorin comprising, consisting essentially of, or consisting of at least 10 contiguous amino acids corresponding to any amino acid sequence within residues 31 to 177 of mammalian Decorin;
i) a polypeptide comprising, consisting essentially of, or consisting of an amino acid sequence corresponding to residues 1 to 170 of mammalian Decorin;
j) a polypeptide comprising, consisting essentially of, or consisting of an amino acid sequence corresponding to residues 31 to 170 of mammalian Decorin;
k) a polypeptide comprising, consisting essentially of, or consisting of an amino acid sequence corresponding to residues 31 to 169 of human Decorin;
l) an N-terminal fragment of mammalian Decorin comprising, consisting essentially of, or consisting of at least 10 contiguous amino acids corresponding to any amino acid sequence within residues 1 to 170 of mammalian Decorin;
m) an N-terminal fragment of mammalian Decorin comprising, consisting essentially of, or consisting of at least 10 contiguous amino acids corresponding to any amino acid sequence within residues 31 to 170 of mammalian Decorin;
n) a polypeptide comprising, consisting essentially of, or consisting of the amino acid sequence depicted in any one of Sequence ID No.s: 1 to 4;
o) a polypeptide comprising, consisting essentially of, or consisting of at least about 10 contiguous amino acids from any one of a) to n) above;
p) a polypeptide comprising or consisting of at least about 10 contiguous amino acids from any one of a) to o) above, wherein said polypeptide comprises a motif or region capable of interacting with or recruiting one or more cells, including one or more stem cells;
q) a polypeptide comprising or consisting of at least about 10 contiguous amino acids from any one of a) to p) above, wherein said polypeptide comprises a motif or region capable of interacting with or recruiting one or more mesenchymal stem cells;
r) a functional fragment, a functional variant, a peptide analogue or peptidomimetic, or a derivative of any one of a) to q) above;
s) a polypeptide having at least about 90% amino acid identity to any one of a) to r) above;
t) any combination of any two or more of a) to s) above.

9. A method of mediating a biological effect in a subject suffering from or susceptible to tissue ischemia, the method comprising administering to the subject an effective amount of a polypeptide as defined in claim 8.

10. A method of modulating tissue repair in a subject suffering from or susceptible to tissue ischemia, the method comprising administering to the subject a therapeutically effective amount of a polypeptide as defined in claim 8.

11. A method of modulating stem cell recruitment or a related process in a subject suffering from or susceptible to tissue ischemia, the method comprising administering to the subject a therapeutically effective amount of a polypeptide as defined in claim 8.

12. The method according to claim 11, wherein the related process is selected from the group consisting of angiogenesis, hematopoiesis, protein expression, induction, or deposition, tissue remodelling, repair, or regeneration, cellular proliferation, cellular differentiation including stem cell differentiation, cellular regulation, apoptosis, modulation of one or more immune responses, modulation of tumourigenesis, chemotaxis, or cell recruitment.

13. The method of any one of claims 9 to 12 wherein the subject has or has an increased risk of having or is predicted to have an increased risk of having a disease or condition associated with tissue ischemia.

14. A pharmaceutical composition for treating or preventing tissue ischemia and/or a disease or condition associated with tissue ischemia, wherein the pharmaceutical composition comprises one or more of the polypeptides as defined in claim 8.

15. A pharmaceutical composition for treating or preventing myocardial infarction, heart failure, and/or cardiomyopathy, wherein the pharmaceutical composition comprises one or more of the polypeptides as defined in claim 8.

16. A polypeptide as defined in claim 8 for treating tissue ischemia and/or a disease or condition associated with tissue ischemia in a subject in need thereof.

17. A polypeptide as defined in claim 8 for treating myocardial infarction, heart failure, and/or cardiomyopathy in a subject in need thereof.

18. A polypeptide as defined in claim 8 for mediating a biological effect in a subject, for modulating stem cell recruitment or a related process in a subject, or for modulating tissue repair in a subject, wherein the subject is suffering from or susceptible to tissue ischemia and/or a disease or condition associated with tissue ischemia.

19. Use of a polypeptide as defined in claim 8 in the preparation of a medicament for the treatment of tissue ischemia and/or a disease or condition associated with tissue ischemia.

20. Use of a polypeptide as defined in claim 8 in the preparation of a medicament for the treatment of myocardial infarction, heart failure, and/or cardiomyopathy.

21. Use of a polypeptide as defined in claim 8 in the preparation of a medicament for mediating a biological effect in a subject, for modulating stem cell recruitment or a related process in a subject, or for modulating tissue repair in a subject, wherein the subject is suffering from or susceptible to tissue ischemia and/or a disease or condition associated with tissue ischemia.

22. The method, composition, polypeptide or use as claimed in any preceding claim, wherein the polypeptide comprises, consists essentially of, or consists of an amino acid sequence corresponding to the amino acid sequence depicted in Sequence ID No.: 6 or 7.

23. The method, composition, polypeptide or use as claimed in any preceding claim, wherein the polypeptide comprises, consists essentially of, or consists of an amino acid sequence corresponding to the amino acid sequence depicted in Sequence ID No.: 2.

24. The method, composition, polypeptide or use as claimed in any preceding claim, wherein the polypeptide comprises, consists essentially of, or consists of an amino acid sequence corresponding to the amino acid sequence depicted in any one of Sequence ID No.s: 3, 4, 9, 10, 12, or 13.
